(19) United States
(12) Patent Application Publication
(10) Pub. No.: US 2024/0228987 A1
(43) Pub. Date: Jul. 11, 2024

(54) HEXOKINASE-DERIVED PEPTIDES AND THERAPEUTICAL USES THEREOF

(71) Applicants: INSERM (INSTITUT NATIONAL DE LA SANTÉ ET DE LA RECHERCHE MÉDICALE), Paris (FR); UNIVERSITÉ DE MONTPELLIER, Montpellier (FR); UNIVERSITE DE PERPIGNAN VIA DOMITIA, Perpignan (FR); ECOLE PRATIQUE DES HAUTES ETUDES, Paris (FR); CENTRE NATIONAL DE LA RECHERCHE SCIENTIFIQUE, Paris (FR)

(72) Inventors: Nicolas TRICAUD, Corbeil Essonnes (FR); Nicolas INGUIMBERT, Perpignan (FR); Benoît GAUTIER, Montpellier Cedex 5 (FR)

(21) Appl. No.: 18/563,078

(22) PCT Filed: May 25, 2022
(86) PCT No.: PCT/EP2022/064320

§ 371 Date: Nov. 21, 2023

(30) Foreign Application Priority Data
May 26, 2021 (EP) 21305690.6

(51) Int. Cl. C12N 9/12; A61K 38/00; A61P 25/28

(52) U.S. Cl. CPC C12N 9/1205; A61P 25/28; A61K 38/00; C12Y 207/01001

(57) ABSTRACT

The inventors previously demonstrated that mitochondrial VDAC1 directly induces Schwann cell demyelination via MAPK and c-jun activation after sciatic nerve injury and diabetic neuropathy and CMT1A. They found that reduction of mitochondrial calcium release by VDAC1 blocking strongly reduces the number of demyelinating Schwann cell in vivo and improve nerve conduction and neuromuscular activity in diabetic, Guillain-Barre syndrome and Charcot-Marie Tooth disease models. Herein, the inventors precisely map the binding region of the N-terminal HK-1 helix through an ala scan completed by a deletion study. Furthermore, they optimized the HK-derived peptide through stabilization of the helix by replacement of non-essential amino acids by the a-aminoisobutyric acid (Aib) known as a helix inducer. Additionally, they described an in-house cellular screening assay based on the ability of MJ to detach HK from VDAC that allows to determine the peptide potency. Overall, their data confirm that N-terminal HK derived peptides acting on VDAC are promising tools for the study of the demyelination process. Thus, the present invention refers to optimized HK-derived peptide and its use for treating peripheral demyelinating disease, myocardium diseases^{10 11}, cancer^{12,13-15}, diabetes^{14 14-16}, lupus-like diseases¹⁷, non-alcoholic fatty liver disease^{24,25}, chemoinduced neuropathy9 Alzheimer disease^{18 19}, Parkinson disease²⁰, Huntington disease²¹, ALS^{22,23} and more generally all neurodegenerative diseases linked to a protein aggregation²⁸.

### FIELD OF THE INVENTION

The present invention relates to hexokinase (HK-) derived peptides and their therapeutic uses, in particular for treating peripheral demyelinating diseases or neurodegenerative diseases or cancer.

### BACKGROUND OF THE INVENTION

The voltage dependent anion channel (VDAC) present at the outer mitochondrial membrane (OMM) is essential for the exchange of ions¹ and metabolites between the mitochondria and cytosolic cell compartment.² VDAC is a transmembrane protein adopting a b-barrel structure with a N-terminal helix lying perpendicular to the pore wall that influences the channel permeability suggesting a molecular gating function.^{3,4,5} Movement of VDAC N-terminal helix allows to switch between an open and closed state that impacts cell homeostasis. It also regulates VDAC multimerization that triggers apoptosis by releasing into the cytosol cytochrome C and calcium and activates caspases.^{6,7} Furthermore, VDAC is a privileged docking site for up to 200 proteins^{8,9} some of them being involved in several pathologies⁹ including myocardium diseases^{10,11}, cancer^{12,13-15}, diabetes^{14,14-16}, lupus-like diseases¹⁷, Alzheimer disease^{18,19}, Parkinson disease²⁰, Huntington disease²¹, ALS^{22,23}, non-alcoholic fatty liver disease^{24,25} and chemoinduced neuropathy⁹. Therefore, VDAC constitutes a therapeutic target and drugs able to modulate its permeability or to disrupt/reinforce its binding to partner proteins are under scrutiny. Among proteins known to interact with VDAC, hexokinases (HK) I and II are major ligands. Due to the pivotal role of this protein/protein interaction in several diseases, the amino acids responsible for the binding of both isoforms of HK to VDAC have been identified. The binding site is located within the first 20 amino acids of the N-terminus sequence of HK, and more precisely the first 10 are essential.^{23,26} A strong sequence homology is observed between HK1 and HK2 N-terminal region, and a large amount of both HK isoforms is known to localize at the outer mitochondrial membrane (OMM) in cells. The mitochondrial fraction of HKs and HK-VDAC1 complexes were found significantly reduced in neurodegenerative disorder and several misfolded proteins involved in neurodegenerative diseases appear to bind to VDAC^{27,19,28} In this context VDAC1 was also identified as a key player of Schwann cells (SC) demyelination.^{29,30}

The Schwann cells (SC) are responsible of myelin production in peripheral nervous system. These cells wrap the axons and remain associated to protect them and allow the correct and efficient action potential transmission⁴⁶. Unfortunately, hereditary and acquired demyelinating diseases of the peripheral nervous system (PNS) are numerous and affect an increasing number of people⁴⁷. Acquired demyelinating diseases are even more common as they include diabetic peripheral neuropathy⁴⁸, drug-related peripheral demyelinating diseases, leprosy and peripheral demyelinating diseases of inflammatory etiology⁴⁹. Demyelinating peripheral neuropathy is a major complication of diabetes and a cause of considerable morbidity⁵⁰. The chronic form of this neuropathy is characterized by Schwann cell demyelination and axonloss and/or degeneration, resulting in the slowing of nerve conduction velocities^{51,52}. Moreover, it has been reported that at least 50% of diabetic patients develop one or several forms of diabetic neuropathies within 25 years after diagnosis⁵³.

Methyl-jasmonate (MJ) a phytohormone is able to detach HK from VDAC1³¹ and induces a spontaneous demyelination²⁹. On the other hand, silencing VDAC in Schwann cells or treating them with the neuroprotective drug olesoxime^{32,33} that binds to VDAC prevent mitochondrial calcium release and block demyelination²⁹. Consequently, restoring a tight HK/VDAC association is an attractive opportunity to treat several diseases in which VDAC permeability is involved. In this particular context of amyotrophic lateral sclerosis, N-terminal HK-1 derived peptides were reported to interact with VDAC in-vitro and in-cellulo preventing VDAC/SOD1 G93A interaction²³. In addition, in hereditary demyelinating peripheral neuropathy CMT4G a mutation in the 5′ non coding sequence of HK1 promotes the expression of an alternatively spliced isoform that lacks the regular Nterminal of HK1³⁴. In peripheral blood mononuclear cell of CMT4G patients and in HEK293 cells mimicking the disease, this leads to a lack of interaction of HK with VDAC (FIG. 1A-C). The mutant HK1 do not block mitochondrial calcium release through VDAC in HEK293 cells mimicking the disease (FIG. 2). Finally, while a peptide derived from the N-terminal of wild-type HK1 is able to prevent mitochondrial calcium release following MJ treatment (FIG. 3), the peptide derived from the mutant HK1 has no effect (FIG. 3). So, a peptide including the N-terminal region of wild-type HK1 might be used to block the calcium efflux and therefore stop the demyelination process in several peripheral nerve diseases such as CMT4G. This peptide may also be a therapeutic proposal for all the diseases in which VDAC permeability is involved.

Herein, the inventors developed optimized HK-derived peptides with an increased stability and affinity to VDAC, and in particular to VDAC1.

### SUMMARY OF THE INVENTION

The invention relates to an HK-derived peptide comprising the amino acid sequence: AQX₁X₂X₃YYX₄ (SEQ ID NO:1), wherein
X₁ is Leucine (L) or Tryptophane (W)
X₂ is Leucine (L) or Tryptophane (W)
X₃ is Alanine (A), D-isomer Alanine (A_{D}) or α-aminoisobutyric acid (U)
X₄ is Phenylalanine (F), Leucine (L) or Tyrosine (Y).

In particular, the invention is defined by the claims.

### DETAILED DESCRIPTION OF THE INVENTION

The inventors precisely map the binding region of the N-terminal HK-1 helix through an ala scan completed by a deletion study. Furthermore, they optimized the HK-derived peptide through stabilization of the helix by replacement of non-essential amino acids by the a-aminoisobutyric acid (Aib) known as a helix inducer. Additionally, they described an in-house cellular screening assay based on the ability of MJ to detach HK from VDAC that allows to determine the peptide potency. Overall, their data confirm that N-terminal HK derived peptides acting on VDAC are promising tools for the study of the demyelination process.

### Peptide of the Invention

The invention relates to an HK-derived peptide comprising the amino acid sequence: Alanine (A)-Glutamine (Q)-X₁-X₂-X₃-Tyrosine (Y)-Tyrosine (Y)-X₄ (SEQ ID NO:1), wherein
X₁ is Leucine (L) or Tryptophan (W)
X₂ is Leucine (L) or Tryptophan (W)
X₃ is Alanine (D)-isomer Alanine (A_{D}) or α-aminoisobutyric acid (U).
X₄ is Phenylalanine (F), Leucine (L) or Tyrosine (Y).

As used herein the term “Hexokinase” (HK) has its general meaning in the art and refers to an enzyme that phosphorylates hexoses (six-carbon sugars), forming hexose phosphate. hexokinases I and II are two isoform of hexokinase and are the main ligands of VDAC.

As used herein, the term “VDAC” has its general meaning in the art and refers to the voltage-dependent anion-selective channel protein 1. VDAC is a major component of the outer mitochondrial membrane, which facilitates the exchange of metabolites and ions across the outer mitochondrial membrane and may regulate mitochondrial functions and cell physiology and differentiation. This protein also forms multimeric channels in the plasma membrane and may be involved in apoptosis and transmembrane electron transport. Alternate splicing results in multiple transcript variants. VDAC has numerous binding partners that controls its permeance and in particular hexokinase (HK). HK binding to VDAC reduces the permeability of the pore notably to calcium. VDAC include the three VDAC isoforms: VDAC1, VDAC2 and VDAC3.

As used herein, the term “peptide” corresponds to the chemical agents belonging to the protein family. A peptide is composed of a mixture of several amino acids. Depending on the number of amino acids involved, peptides are categorized as dipeptides, composed of 2 amino acids, tripeptides, made up of 3 amino acids, and so on. Peptides composed of more than 10 amino acids are called polypeptides. Thus, the peptide of the invention can be considered as a polypeptide.

The peptides according to the invention, may be produced by conventional automated peptide synthesis methods or by recombinant expression. General principles for designing and making proteins are well known to those of skill in the art.

Peptides of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. Peptides of the invention may also be synthesized by solid-phase technology employing an exemplary peptide synthesizer. The purity of any given protein; generated through automated peptide synthesis or through recombinant methods may be determined using reverse phase HPLC analysis. Chemical authenticity of each peptide may be established by any method well known to those of skill in the art. As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides. A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. U.S. Pat. Nos. 6,569,645; 6,043,344; 6,074,849; and 6,579,520 provide specific examples for the recombinant production of peptides and these patents are expressly incorporated herein by reference for those teachings. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

As used herein, the term “amino acid” refers to natural or unnatural amino acids in their D and L stereoisomers for chiral amino acids. It is understood to refer to both amino acids and the corresponding amino acid residues, such as are present, for example, in peptidyl structure. Natural and unnatural amino acids are well known in the art. Common natural amino acids include, without limitation, alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). Uncommon and unnatural amino acids include, without limitation, α-aminoisobutyric acid (Aib, U), allyl glycine (AllylGly), norleucine (Nle), norvaline, biphenylalanine (Bip), citrulline (Cit), 4-guanidinophenylalanine (Phe(Gu)), homoarginine (hArg), homolysine (hLys), 2-naphtylalanine (2-Nal), ornithine (Orn), Cyclohexylalanine (Cha, Fx), and pentafluorophenylalanine.

In some embodiments, the HK-derived peptide of the invention comprises 7, 8, 9, 10, 11, 12, 13, 14, 15 or 16 amino acids.

In some embodiments, the HK-derived peptide of the invention does not consist of the amino sequence: Alanine (A)-Glutamine (Q)-Leucine (L)-Leucine (L)-Alanine (A)-Tyrosine (Y)-Tyrosine (Y)-Phenylalanine (F) (SEQ ID NO:95).

In some embodiments, the HK-derived peptide of the invention does not consist or comprise the amino sequence of Alanine (A)-Alanine (A)-Glutamine (Q)-Leucine (L)-Leucine (L)-Alanine (A)-Tyrosine (Y)-Tyrosine (Y)-Phenylalanine (F)-Threonine (T)-Glutamic acid (E)-Leucine (L)-Lysine (K) (SEQ ID NO:96).

In some embodiment, the HK-derived peptide comprises the amino acid sequence: Alanine (A)-Glutamine (Q)-X₁-X₂-X₃-Tyrosine (Y)-Tyrosine (Y)-X₄-Threonine (T)-Glutamic acid (E)-X₅-Lysine (K) (SEQ ID NO:2), wherein
X₁ is Leucine (L) or Tryptophan (W)
X₂ is Leucine (L) or Tryptophan (W)
X₃ is Alanine (A), D-isomer Alanine (A_{D}) or α-aminoisobutyric acid (U)
X₄ is Phenylalanine (F), Leucine (L) or Tyrosine (Y).
X₅ is Leucine (L) or Tryptophan (W).

In some embodiment, X₃ is α-aminoisobutyric acid (U).

In some embodiment, the HK-derived peptide comprises or consists of the amino sequence in table 1.

TABLE 1
Optimized HK-1 derived peptide.

| HK-derived peptide | SEQ ID NO: | SEQUENCE: |
|---|---|---|
| 5i | 3 | AQLLAYYLTEWK |
| 5j | 4 | AQLLAYYYTEWK |
| 5k | 5 | AQWLAYYFTEWK |
| 5l | 6 | AQWLAYYLTELK |
| 5m | 7 | AQWLAYYYTELK |
| 5n | 8 | AQWLAYYLTEWK |
| 5o | 9 | AQWLAYYYTEWK |
| 5p | 10 | AQLWAYYFTEWK |
| 5q | 11 | AQLWAYYLTELK |
| 5r | 12 | AQLWAYYYTELK |
| 5s | 13 | AQLWAYYLTEWK |
| 5t | 14 | AQLWAYYYTEWK |
| 5u | 15 | AQWWAYYFTELK |
| 5v | 16 | AQWWAYYLTELK |
| 5w | 17 | AQWWAYYYTELK |
| 5x | 18 | AQWWAYYFTEWK |
| 5y | 19 | AQWWAYYLTEWK |
| 5z | 20 | AQWWAYYYTEWK |
| 6l | 21 | NleIAAQWLAYYL |
| 6m | 22 | NleIAAQWLAYYY |
| 6n | 23 | NleIAAQLWAYYL |
| 6o | 24 | NleIAAQLWAYYY |
| 6p | 25 | NleIAAQWWAYYF |
| 6q | 26 | NleIAAQWWAYYL |
| 6r | 27 | NleIAAQWWAYYY |
| 7a′ | 28 | AQLLUYYFTELK |
| 7g′ | 29 | AQWWUYYFTEWK |
| 5i-U | 30 | AQLLUYYLTEWK |
| 5j-U | 31 | AQLLUYYYTEWK |
| 5k-U | 32 | AQWLUYYFTEWK |
| 5l-U | 33 | AQWLUYYLTELK |
| 5m-U | 34 | AQWLUYYYTELK |
| 5n-U | 35 | AQWLUYYLTEWK |
| 5o-U | 36 | AQWLUYYYTEWK |
| 5p-U | 37 | AQLWUYYFTEWK |
| 5q-U | 38 | AQLWUYYLTELK |
| 5r-U | 39 | AQLWUYYYTELK |
| 5s-U | 40 | AQLWUYYLTEWK |
| 5t-U | 41 | AQLWUYYYTEWK |
| 5u-U | 42 | AQWWUYYFTELK |
| 5v-U | 43 | AQWWUYYLTELK |
| 5w-U | 44 | AQWWUYYYTELK |
| 5y-U | 45 | AQWWUYYLTEWK |
| 5z-U | 46 | AQWWUYYYTEWK |
| 6l-U | 47 | NleIAAQWLUYYL |
| 6m-U | 48 | NleIAAQWLUYYY |
| 6n-U | 49 | NleIAAQLWUYYL |
| 6o-U | 50 | NleIAAQLWUYYY |
| 6p-U | 51 | NleIAAQWWUYYF |
| 6q-U | 52 | NleIAAQWWUYYL |
| 6r-U | 53 | NleIAAQWWUYYY |

In some embodiment, the HK-derived peptide comprises or consists of the amino sequence SEQ ID NO: 18, SEQ ID NO:20, SEQ ID NO:25, SEQ ID NO: 26, SEQ ID NO:28 or SEQ DI NO:29.

Moreover, the inventors demonstrated the importance of the AUAU patch or the 3-CF₃-Ph[Tz]Aib fused in N-terminal to enhance the stability of the HK-derived peptide. (see FIG. 7). The inventors also demonstrated the importance of the substitution of the second alanine by α-aminoisobutyric acid to enhance the stability of the HK-derived peptide.

The inventors show in previous study that 3-CF₃Ph[Tz]U dipeptide as N-terminal capping enhance peptide insertion within membrane (see FIG. 4A and Das et al, Chemistry. 2017 Dec. 24).

As used herein, the term 3-CF₃Ph[TZ]U dipeptide has its general meaning in the art and refers to a 2-methyl-2-{4-[(3-trifluoromethyl)phenyl]-1H-1,2,3-triazol-1yl}propanoic acid, also known as 1,4-disubstituted-1,2,3-triazole coupled to an α-aminoisobutyric acid with the following formula C₁₆H₁₆F₃N₃O:

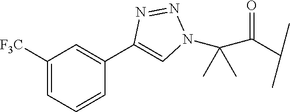 [SMILES: CC(C)C(=O)C(C)(C)n1cc(-c2cccc(C(F)(F)F)c2)nn1]

In some embodiment, a sequence AUAU (SEQ ID NO:54) or AU (SEQ ID NO:55) is coupled to the HK-derived peptide.

In some embodiment, a sequence AUAU (SEQ ID NO:54) or AU (SEQ ID NO:55) is coupled in N-terminal of the HK-derived peptide.

In some embodiment, a sequence AUAU (SEQ ID NO:54) or AU (SEQ ID NO:55) is coupled in C-terminal of the HK-derived peptide.

In some embodiment, the dipeptide 3-CF₃Ph[Tz]U is coupled in N-terminal of the HK-derived peptide.

In some embodiment, a cell penetrating sequence is coupled to the HK-derived peptide.

As used herein, the term “cell penetrating sequence” has its general meaning in the art and refers to short sequence that facilitate cellular intake and uptake of the peptide of the invention. Based on the origin of peptides, CPPs are divided into chimeric, protein-derived and synthetic. Cell penetrating sequence include but are not limited to Penetratin, octaarginine (R8), tat, Transportan and Xentry. Penetratin is a cell penetrating peptide from the first generation, which is derived from Drosophila Antennapedia Homeodomain. Penetratin overcomes the plasma membrane barrier of mammalian cells through the macropinocytotic pathway and efficiently delivers molecular cargoes in a biologically active form. The tat peptide is derived from the transactivator of transcription (tat) of human immunodeficiency virus. TAT is an arginine-rich peptide which directly penetrates plasma membrane and stabilized DNA. Transportan is a chimeric CPP, which derived from galanin and mastoparan. Xentry is a short-peptide derived from an N-terminal region of the X-protein of the hepatitis B virus. Xentry permeates adherent cells using syndecan-4 as a portal for entry. Horton peptide is a synthetic cell-permeable peptide that are able to enter mitochondria. The sequences of the MPPs were designed to display two properties known to be important for passage across both the plasma and mitochondrial membranes: positive charge and lipophilic character as explained in Horton et al, Chem Biol. 2008⁵⁸.

In some embodiment, the cell penetrating sequence is coupled in N-terminal or C-terminal of the HK-derived peptide.

In some embodiment, the cell penetrating sequence consists of the sequence in table 2:

TABLE 2
Cell penetrating sequence

| Cell penetrating | SEQ | |
|---|---|---|
| sequence | ID NO: | Sequence |
| Penetratin | 56 | RQIKIWFQNRRMKWKK |
| R8 | 57 | RRRRRRRR |
| tat | 58 | GRKKRRQRRRPQ |
| Transportan | 59 | GWTLNSAGYLLGKINLKALAALAKKIL |
| Xentry | 60 | LCLRPVG |
| Horton Peptide | 61 | FₓRFₓRFₓRFₓR |

In some embodiment, the cell penetrating sequence is tat (SEQ ID NO:58)

In some embodiment, a sequence AUAU (SEQ ID NO:54) or AU (SEQ ID NO:55) is coupled in N-terminal of the HK-derived peptide and a cell penetrating sequence is coupled in C-terminal of the HK-derived peptide.

In some embodiment, a sequence AUAU (SEQ ID NO:54) or AU (SEQ ID NO:55) is coupled in C-terminal of the HK-derived peptide and a cell penetrating sequence is coupled in N-terminal of the HK-derived peptide.

In some embodiment, the dipeptide 3-CF₃Ph[Tz]U is coupled in N-terminal of the HK-derived peptide and a cell penetrating sequence is coupled in C-terminal of the HK-derived peptide.

In some embodiment, the HK-derived peptide comprises or consists of the amino sequence SEQ ID NO: 18, SEQ ID NO:20, SEQ ID NO:25, SEQ ID NO:26, SEQ ID NO:28 or SEQ DI NO:29, wherein a dipeptide 3-CF₃Ph[Tz]U is coupled in N-terminal of the HK-derived peptide.

In some embodiment, the HK-derived peptide comprises or consists of the amino sequence SEQ ID NO: 18, SEQ ID NO:20, SEQ ID NO:25, SEQ ID NO:26, SEQ ID NO:28 or SEQ DI NO:29, wherein a sequence AUAU (SEQ ID NO:54) or a sequence AU (SEQ ID NO:55) is coupled in N-terminal of the HK-derived peptide.

In some embodiment, the HK-derived peptide comprises or consists of the amino sequence SEQ ID NO: 18, SEQ ID NO:20, SEQ ID NO:25, SEQ ID NO:26, SEQ ID NO:28 or SEQ DI NO:29, wherein a dipeptide 3-CF₃Ph[Tz]U is coupled in N-terminal of the HK-derived peptide and a cell penetrating sequence is coupled in C-terminal of the HK-derived peptide.

In some embodiment, the HK-derived peptide comprises or consists of the amino sequence SEQ ID NO: 18, SEQ ID NO:20, SEQ ID NO:25, SEQ ID NO:26, SEQ ID NO:28 or SEQ DI NO:29, wherein a sequence AUAU (SEQ ID NO:54) or a sequence AU (SEQ ID NO:55) is coupled in N-terminal of the HK-derived peptide and a cell penetrating sequence is coupled in C-terminal of the HK-derived peptide.

In some embodiment, the HK-derived peptide comprises or consists of the amino sequence SEQ ID NO: 18, SEQ ID NO:20, SEQ ID NO:25, SEQ ID NO:26, SEQ ID NO:28 or SEQ DI NO:29, wherein a sequence AUAU (SEQ ID NO:54) or a sequence AU (SEQ ID NO:55) is coupled in C-terminal of the HK-derived peptide and a cell penetrating sequence is coupled in N-terminal of the HK-derived peptide.

In some embodiment, the cell penetrating sequence is tat (SEQ ID NO:58) In a second aspect, the invention relates to a vector that includes the HK-derived peptide of the present invention.

Typically, the peptide may be delivered in association with a vector. The HK-derived peptide of the present invention is included in a suitable vector, such as a plasmid, cosmid, episome, artificial chromosome, phage or a viral vector. So, a further object of the invention relates to a vector comprising the peptide of the invention. Typically, the vector is a viral vector, which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, AAVrh10 and mutated forms thereof. AAV vectors can have one or more of the AAV wild-type genes deleted in whole or part, preferably the rep and/or cap genes, but retain functional flanking ITR sequences. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein that allows transduction of cells of human and other species. Typically, the nucleic acid molecule or the vector of the present invention include “control sequences'”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In some embodiment, the vector is an adeno-associated virus (AAV).

In some embodiment, the vector is AAV9 or AAVrh10.

### Therapeutics Methods

The formation of the myelin sheath around peripheral nerve axons by Schwann cells is essential for the rapid propagation of action potentials. Several peripheral neuropathies have as pathological physiology a process of demyelination. The inventors previously demonstrated that mitochondrial VDAC1 directly induces Schwann cell demyelination via MAPK pathways and c-jun activation after sciatic nerve injury, diabetic neuropathy and CMT1A. They found that reduction of mitochondrial calcium release by VDAC1 blocking strongly reduces the number of demyelinating Schwann cell in vivo and improve nerve conduction and neuromuscular activity in diabetic, Guillain-barré syndrome and Charcot-Marie Tooth disease models.

Consequently, restoring a tight HK/VDAC association is an attractive opportunity against different peripheral demyelinating disease and all other diseases where VDAC permeability is involved.

Accordingly, the present invention relates to the HK-derived peptide or the vector of the invention for use as drugs.

In other words, the present invention relates to the HK-derived peptide or the vector of the invention for use in therapy.

In more particular, the invention relates to the HK-derived peptide or the vector of the invention of the invention for use in the treatment of peripheral demyelinating disease.

In other words, the present invention relates to a method of treating a peripheral demyelinating disease in a subject in need thereof comprising administering to the subject a therapeutically effective amount of the HK-derived peptide of the invention or the vector of the invention.

As used herein, the term “subject” refers to a human or another mammal (e.g., mouse, rat, rabbit, hamster, dog, cat, cattle, swine, sheep, horse or primate). In some embodiments, the subject is a human being. Typically, the subject is affected or likely to be affected with a disease affecting the peripheral nervous system. Typically, the subject is affected or likely to be affected with a peripheral demyelinating disease.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, a “therapeutically effective amount” is intended for a minimal amount of active agent (i.e the peptides of the invention) which is necessary to impart therapeutic benefit to a patient. For example, a “therapeutically effective amount of the active agent” to a patient is an amount of the active agent that induces, ameliorates or causes an improvement in the pathological symptoms, disease progression, or physical conditions associated with the disease affecting the patient. It will be understood that the total daily usage of the compounds and compositions of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts.

As used herein, the term “peripheral demyelinating diseases” has its general meaning in the art and refers to a spectrum of disorders that involve substantial damage to axons and glial cells, particularly schwann cells (SC) in the peripheral nervous system (PNS). The wide variety of morphologies exhibited by peripheral demyelinating diseases can each be uniquely attributed to an equally wide variety of causes. For instance, peripheral demyelinating diseases can be genetically acquired (“hereditary peripheral demyelinating diseases”), or can result from a systemic disease, or can be induced by a toxic agent or an infectious agent (“acquired peripheral demyelinating diseases”).

The method of the present invention has wide applicability to the treatment or prophylaxis of peripheral demyelinating diseases affecting the regulation of peripheral nerves including peripheral ganglionic neurons, sympathetic, sensory neurons, and myelinated motor and sensory neurons.

In particular, the method of the present invention is useful in treatments designed to rescue, for example, eyes nerves, inner ear and accoustical nerves, and myelinated motor and sensory neurons. In particular, the method of the present invention is particularly suitable for preventing peripheral nerve demyelination.

The peptides of the present invention is suitable for the treatment of hereditary peripheral demyelinating diseases.

Hereditary peripheral demyelinating diseases are caused by genetic abnormalities which are transmitted from generation to generation. For several of these, the genetic defect is known, and tests are available for diagnosis and prenatal counseling. In particular, the diagnosis of a hereditary peripheral demyelinating disease is usually suggested with the early onset of neuropathic symptoms, especially when a positive family history is also present. Prior to the recent genetic advances, the diagnosis was supported by typical findings of marked slowing of the nerve conduction studies on electromyography and a nerve biopsy. Typical findings on a nerve biopsy include the presence of so-called onion-bulbs, indicating a recurring demyelinating and remyelinating of the nerve fibers. There are several hereditary neuropathies that are related directly or indirectly to peripheral nerve demyelination. Examples include but are not limited to Refsum's disease, Abetalipoproteinemia, Tangier disease, Krabbe's disease, Metachromatic leukodystrophy, Charcot-Marie-Tooth (CMT) disease, Fabry's disease, Hereditary Neuropathy with liability to pressure palsies (HNPP), Familial Amyloidotic Neuropathy, Hereditary sensory neuropathy Type II (HSN II), hereditary porphyria, muscular dystrophies such as congenital muscular dystrophy 1A, and Dejerine-Sottas syndrome.

In some embodiment, the hereditary demyelinating diseases is Charcot-Marie-Tooth (CMT) Diseases.

CMT disease are the most common hereditary neurological disorders. It is characterized by weakness and atrophy of muscles due to segmental demyelination of peripheral nerves and associated degeneration of axons and anterior horn cells. During the last 15 years, there has been a substantive increase in knowledge about the genetic basis of Charcot-Marie-Tooth disease (CMT) with over 60 genes known at present. A regularly updated list can be found at http://www.molgen.ua.ac.be/CMTMutations/Home/IPN.cfm. Autosomal dominant inheritance is usual, and associated degenerative CNS disorders, such as Friedreich's ataxia, are common. In some embodiments, the peptides of the present invention can be used for the treatment of Charcot-Marie-Tooth disease type 4G and 1A.

The peptides of the present invention is also suitable for the treatment of acquired peripheral demyelinating diseases.

Acquired peripheral demyelinating diseases has its general meaning in the art and include but are not limited to diabetic neuropathies, immune-mediated neuropathies; acute and chronic motor neuropathy; acute and chronic sensory neuropathy; acute and chronic autonomic system neuropathy; miller-fisher syndrome which there is paralysis of eye gaze, incoordination, and unsteady gait;

In some embodiments, the peptide of the invention is used in the treatment of diabetic neuropathies. Diabetes is the most common known cause of neuropathy. It produces symptoms in approximately 50% of people with diabetes. In most cases, the neuropathy is predominantly sensory, with pain and sensory loss in the hands and feet. But some diabetes patients have chronic demyelinating neuropathy, mononeuritis or mononeuritis multiplex which causes weakness in one or more nerves, or lumbosacral plexopathy or amyotrophy which causes weakness in the legs, inflammation, necrosis and abscess.

In some embodiments, the peptide of the invention is used in the treatment of immune-mediated neuropathies. The main function of the immune system is to protect the body against infectious organisms which enter from outside. In some cases, however the immune system turns against the body and causes autoimmune disease. The immune system consists of several types of white blood cells, including T-lymphocytes, which also regulate the immune response; and B-lymphocytes or plasma cells, which secrete specialized proteins called “antibodies” Sometimes, for unknown reasons, the immune system mistakenly attacks parts of the body such as the peripheral nerves. This is “autoimmune” Peripheral Neuropathy. There are several different types, depending on the part of the peripheral nerve which is attacked and the type of the immune reaction. For instance, the method of the present invention is suitable for treating Guillain-Barre Syndrome (GBS). An acute neuropathy because it comes on suddenly or rapidly. Guillain-Barre Syndrome can progress to paralysis and respiratory failure within days or weeks after onset. The neuropathy is caused when the immune system destroys the myelin sheaths of the motor and sensory nerves. It is often preceded by infection, vaccination or trauma, and that is thought to be what triggers the autoimmune reaction. The disease is self-limiting, with spontaneous recovery within six to eight weeks. But the recovery is often incomplete.

An another acquired peripheral demyelinating disease which is may be treated by the peptide of the present invention is Chronic Inflammatory Demyelinating Polyneuropathy (CIDP). CIDP is thought to be a chronic and more indolent form of the Guillain-Barre Syndrome. The disease progresses either with repeated attacks, called relapses, or in a stepwise or steady fashion. As in GBS, there appears to be destruction of the myelin sheath by antibodies and T-lymphocytes. But since there is no specific test for CIDP, the diagnosis is based on the clinical and laboratory characteristics.

Chronic Polyneuropathies with antibodies to peripheral nerves is an another acquired peripheral demyelinating diseases for which the peptide of the present inventions can be used. In some types of chronic neuropathies, antibodies to specific components of nerve have been identified. These include demyelinating peripheral disease associated with antibodies to the Myelin Associated Glycoprotein (MAG), motor neuropathy associated with antibodies to the gangliosides GM1 or GD1a, and sensory neuropathy associated with anti-sulfatide or GD1b ganglioside antibodies. The antibodies in these cases bind to oligosaccharide or sugar like molecules, which are linked to proteins (glycoproteins) or lipids (glycolipids or gangliosides) in the nerves.

The peptide of the present invention can also be used for treating peripheral demyelinating diseases associated with vasculitis or inflammation of the blood vessels in peripheral nerves. Peripheral demyelinating disease can also be caused by Vasculitis—an inflammation of the blood vessels in peripheral nerve. It produces small “strokes” along the course of the peripheral nerves, and may be restricted to the nerves or it may be generalized, include a skin rash, or involve other organs. Several rheumatological diseases like Rheumatoid Arthritis, Lupus, Periarteritis Nodosa, or Sjogren's Syndrome, are associated with generalized Vasculitis, which can also involve the peripheral nerves. Vasculitis can cause Polyneuritis, Mononeuritis, or Mononeuritis Multiplex, depending on the distribution and severity of the lesions.

In some embodiments, the method of the present invention is suitable for the treatment of peripheral demylinating diseases associated with monoclonal gammopathies. In Monoclonal Gammopathy, single clones of B-cells or plasma cells in the bone marrow or Iymphoid organs expand to form benign or malignant tumors and secrete antibodies. “Monoclonal” is because there are single clones of antibodies. And “Gammopathy” stands for gammaglobulins, which is another name for antibodies. In some cases, the antibodies react with nerve components; in others, fragments of the antibodies form amyloid deposits.

In some embodiments, the method of the present invention is suitable for the treatment of peripheral demyelinating diseases associated with tumors or neoplasms. Neuropathy can be due to direct infiltration of nerves by tumor cells or to indirect effect of the tumor. The latter is called Paraneoplastic Neuropathy. Several types have been described. For instance, the method of the present inventions can be used to manage sensory neuropathy associated with lung cancer. Likewise, the method of the present invention can be used to treat peripheral demyelinating diseases associated with multiple myeloma. In some embodiments, the method of the present invention is suitable for the treatment of peripheral demyelinating diseases associated with Waldenstrom's Macroglobulemia, Chronic Lymphocytic Leukemia, or B-cell Lymphoma. In some embodiments, the method of the present invention is used as part of therapeutic protocol for the treatment of patients with cancers where peripheral demyelinating disease is a consequence of local irradiation or be caused by a chemotherapeutic agent. Chemotherapeutic agents known to cause sensory and/or motor neuropathies include vincristine, an antineoplastic drug used to treat haematological malignancies and sarcomas, as well as cisplatin, taxol and others. The neurotoxicity is dose-related, and exhibits as reduced intestinal motility and peripheral neuropathy, especially in the distal muscles of the hands and feet, postural hypotension, and atony of the urinary bladder. Similar problems have been documented with taxol and cisplatin (MoUman, J. E., 1990, New Eng Jour Med. 322:126-127), although cisplatin-related neurotoxicity can be alleviated with nerve growth factor (NGF) (Apfel, S. C. et al, 1992, Annals of Neurology 31:76-80). Although the neurotoxicity is sometimes reversible after removal of the neuro toxic agent, recovery can be a very slow process (Legha, S., 1986, Medical Toxicology 1:421-427; Olesen, et al, 1991, Drug Safety 6:302-314).

In some embodiments, the method of the present invention is suitable for the treatment of peripheral demyelinating diseases caused by a drug such as Chloroquine, FK506 (Tacrolimus), Perhexiline, Procainamide and Zimeldine.

In some embodiments, the method of the present invention is suitable for the treatment of peripheral demyelinating diseases caused by infections. Peripheral demyelinating diseases can be caused by infection of the peripheral nerves. Viruses that cause peripheral demyelinating diseases include the AIDS virus, HIV-I, which causes slowly progressive sensory neuropathy, Cytomegalovirus which causes a rapidly progressive paralytic neuropathy, Herpes Zoster which cause Shingles, and Poliovirus which causes a motor neuropathy. Hepatitis B or C infections are sometimes associated with vasculitic neuropathy. Bacterial infections that cause neuropathy include Leprosy which causes a patchy sensory neuropathy, and Diphtheria which can cause a rapidly progressive paralytic neuropathy. Other infectious diseases which causes neuropathy include Lyme disease which is caused by a spirochete, and Trypanosomiasis which is caused by a parasite. Both commonly present with a multifocal neuropathy.

In some embodiments, the peptide of the present invention is suitable for the treatment of peripheral demyelinating diseases caused by nutritional imbalance. Deficiencies of Vitamins B12, B1 (thiamine), B6 (pyridoxine), or E, for example, can produce polyneuropathies with degeneration of peripheral nerve axons. This can be due to poor diet, or inability to absorb the nutrients from the stomach or gut. Moreover, megadoses of Vitamin B6 can also cause a peripheral demyelinating disease, and the peptide of the present invention can be used as part of a de-toxification program in such cases.

In some embodiments, the peptide of the present invention is suitable for the treatment of peripheral demyelinating diseases arising in kidney diseases. Chronic renal failure can cause a predominantly sensory peripheral neuropathy with degeneration of peripheral nerve axons.

In some embodiments, the peptide of the present invention is suitable for the treatment of hypothyroid neuropathies. Hypothyroidism is sometimes associated with a painful sensory polyneuropathy with axonal degeneration. Mononeuropathy or Mononeuropathy Multiplex can also occur due to compression of the peripheral nerves by swollen tissues.

In some embodiments, the peptide of the present invention is suitable for the treatment of peripheral demyelinating diseases caused by Alcohol and Toxins. Certain toxins can cause Peripheral Neuropathy. Lead toxicity is associated with a motor neuropathy; arsenic or mercury cause a sensory neuropathy, Thalium can cause a sensory and autonomic neuropathy, several of the organic solvents and insecticides can also cause polyneuropathy. Alcohol is directly toxic to nerves and alcohol abuse is a major cause of neuropathy. The peptide of the present invention can be used, in some embodiments, as part of a broader detoxification program. In still another embodiment, the peptide of the present invention can be used for the treatment of peripheral demyelinating diseases caused by drugs. Several drugs are known to cause neuropathy. They include, among others, nitrofurantoin, which is used in pyelonephritis, amiodarone in cardiac arrhythmias, disulfiram in alcoholism, ddC and ddl in AIDS, and dapsone which is used to treat Leprosy. As above, the peptide of the present invention can be used, in some embodiments, as part of a broader detoxification program.

In some embodiments, the peptide of the present invention is suitable for the treatment of peripheral demyelinating diseases caused by trauma or compression. Localized neuropathies can result from compression of nerves by external pressure or overlying tendons and other tissues. The best known of these are the Carpal Tunnel Syndrome which results from compression at the wrist, and cervical or lumbar radiculopathies (Sciatica) which result from compression of nerve roots as they exit the spine. Other common areas of nerve compression include the elbows, armpits, and the back of the knees.

The peptide of the present invention is also useful in the treatment of variety of idiopathic peripheral demyelinating diseases. The term “idiopathic” is used whenever the cause of the peripheral demyelinating disease cannot be found. In these cases, the peripheral demyelinating disease is classified according to its manifestations, i.e., sensory, motor, or sensorimotor idiopathic polyneuropathy.

VDAC pore is a privileged docking site of proteins involved in many diseases that made it a therapeutic target for drugs able to disrupt or reinforce its binding to partner proteins. Blocking this channel upstream from the signalization pathway to be activated is of interest in the fight against myocardium diseases^{10,11}, cancer^{12,13-15}, diabetes^{14,14-16}, lupus-like diseases¹⁷, non-alcoholic fatty liver disease^{24,25}, chemoinduced neuropathy⁹ Alzheimer disease^{18,19}, Parkinson disease²⁰, Huntington disease²¹, ALS^{22,23} and more generally all neurodegenerative diseases linked to a protein aggregation²⁸

Accordingly, the invention also relates to the HK-derived peptide of the invention or the vector of the invention for use in the treatment of myocardium diseases, cancer, diabetes, lupus-like diseases, non-alcoholic fatty liver disease, neurodegenerative disease such as chemoinduced neuropathy Alzheimer disease, Parkinson disease, Huntington disease¹ or ALS.

As used herein, the term “lupus-like diseases” hast its general meaning in the art and refers to disorder with clinical, histological, and immunological features similar to idiopathic systemic lupus erythematosus.

As used herein, the term “non-alcoholic fatty liver disease” hast its general meaning in the art and refers to conditions caused by a build-up of fat in the liver. The main stages of NAFLD is a simple fatty liver (steatosis); a non-alcoholic steatohepatitis (NASH) where the liver has become inflamed; a fibrosis where persistent inflammation causes scar tissue around the liver and nearby blood vessels, and cirrhosis—the most severe stage, occurring after years of inflammation, where the liver shrinks and becomes scarred and lumpy.

As used herein, the term “Neurodegenerative disease” has its general meaning in the art and refers to diseases with neurodegeneration which is the progressive loss of structure or function of neurons, including death of neurons. Many neurodegenerative diseases including amyotrophic lateral sclerosis, Parkinson's, Alzheimer's, and Huntington's occur as a result of neurodegenerative processes. Such diseases are incurable, resulting in progressive degeneration and/or death of neuron cells. As research progresses, many similarities appear that relate these diseases to one another on a sub-cellular level. Discovering these similarities offers hope for therapeutic advances that could ameliorate many diseases simultaneously. There are many parallels between different neurodegenerative disorders including atypical protein protein aggreatation as well as induced cell death (Rubinsztein D C (2006). Nature. 443 (7113): 780-6 and Bredesen D E, et al (2006). Nature. 443 (7113): 796-802). In some embodiment, the neurodegenerative diseases is a disease linked to a protein aggregation²⁸

Neurodegenerative diseases include but are not limited to Alzheimer's disease and in particular chemoinduced neuropathy Alzheimer disease^{18,19}, dementia with Lewy bodies (DLB), amyotrophic lateral sclerosis (ALS) with frontotemporal dementia, inclusion body myopathy with Paget's disease of bone and/or frontotemporal dementia (IBMPFD), frontotemporal lobar degeneration, synucleopathies, Huntington's disease and Parkinson's disease, amyloidopathies including amyloid angiopathies, tauopathies including frontotemporal dementia with Parkinsonism linked to chromosome 17, neuromuscular diseases with protein inclusions, as well as developmental diseases including Down syndrome.

In some embodiments, the peptide of the present invention can be used to treat, or at least reduce the severity of chemoinduced neuropathy Alzheimer disease, Parkinson disease, Huntington disease or ALS).

As used herein, the term “diabetes” has its general meaning in the art and refers to a common metabolic disorder characterized by chronic hyperglycaemia. It is associated with greater risk of heart disease, stroke, peripheral neuropathy, renal disease, blindness and amputation. There are three main types of diabetes: type 1 diabetes, type 2 diabetes and gestational diabetes. Previous study demonstrated that VDAC1 inhibition restores R cell function and prevents hyperglycemia in diabetic mice.

As used herein, the term “cancer” has its general meaning in the art and refers to abnormal cell growth with the potential to invade or spread to other parts of the body. Cancer cells share several features that distinguish them from normal cells, including avoiding apoptosis. Defects in the regulation or even evasion of apoptosis are hallmarks of cancer. VDAC1 offers a unique target for anti-cancer therapies because of its role as a key regulator of energy and metabolism and apoptosis⁸. Voltage-dependent anion channel 1 is highly expressed in many cancer types compared to the levels in normal cells.⁸ The peptides of the invention is suitable to treat cancer by interfering with the binding of anti-apoptotic proteins such hexokinase to VDAC thereby permitting apoptosis induction.

According to the invention, the cancer may be selected in the group consisting of adrenal cortical cancer, anal cancer, bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

### Pharmaceutical Composition

The peptide of the invention may be used or prepared in a pharmaceutical composition.

In another aspect, the invention relates to a pharmaceutical composition comprising the peptide of the invention.

The invention relates to the pharmaceutical composition comprising the peptide of the invention or the vector of the invention for use in the treatment of peripheral myelinating disease, myocardium diseases, cancer, diabetes, lupus-like diseases, non-alcoholic fatty liver disease or neurogenerative disease such as chemoinduced neuropathy⁹ Alzheimer disease, Parkinson disease, Huntington disease, ALS.

Typically, the peptide of the invention, may be combined with pharmaceutically acceptable excipients, and optionally sustained-release matrices, such as biodegradable polymers, to form therapeutic compositions.

As used herein, the term “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising inhibitors of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: Amount of VDAC1 that co-immunoprecipitates with HK in Peripheral Blood Mononuclear Cells (PBMC) of patients blood and in HEK293 cells expressing wt HK or CMT4G-mutated HK (or nothing-control). A. PBMC were collected from peripheral blood of CMT4G patients or controls by centrifugation, washed and lysed in a detergent solution to extract proteins. HK was precipitated with a specific monoclonal antibody using sepharose-beads coupled with G protein. After washing, co-immunoprecipitated proteins were analysed through SDS-PAGE and Western blotting using polyclonal antibody against VDAC1. The amount of co-immunoprecipitated VDAC1 was normalized over the amount of the protein in the cell lysate. B. Sequences of the main isoform and of the alternatively spliced AlT2 isoform of human HK1 showing the contribution of Exons 1 and 2 and alternative exons T3 and T4 to the Nterminal sequence of each isoform. See Hantke, J. et al. 2009 C. HEK293 cells were transfected with a plasmid expressing wild-type (wt) Flag-tagged human HK1 or CMT4G-mutated Flag-tagged human HK1. 48h later cells were washed and lysed in a detergent solution to extract proteins. HK was precipitated with a monoclonal anti-Flag antibody using sepharose-beads coupled with G protein. After washing co-immunoprecipitated proteins were analysed through SDS-PAGE and Western blotting using polyclonal antibody against VDAC1. The amount of co-immunoprecipitated VDAC1 was normalized over the amount of the protein in the cell lysate.

FIG. 2: Fluorescence intensity of the mitochondrial calcium probe in HEK293 cells overexpressing wt HK or CMT4G-mutated HK (or nothing-control). HEK 293 cells were transfected with a plasmid expressing mito-GCaMP2, the fluorescent probe detecting calcium in the mitochondrial matrix, alone (Control) or together with a plasmid expressing wt Flag-tagged human HK1 or CMT4G-mutated Flag-tagged human HK1. 48 hours later cells were washed, fixed with paraformaldehyde and treated with DAPI to detect nuclei. GFP fluorescence was recorded using a LSM700 Zeiss confocal microscope and normalized over the background value in each picture.

FIG. 3: Time-lapse recording of fluorescence intensity of the mitochondrial calcium probe in HEK293 cells treated with Methyl Jasmonate (MJ, 6 milliMolar) and Nterminal peptide of wt HK1 (HK1-Nt peptide) or Nterminal peptide of mutated HK1 (HKmut-Nt peptide). HEK 293 cells were transfected with a plasmid expressing mito-GCaMP2. 48 hours later cells were imaged using a Zeiss Axio-observer designed for live-imaging and treated with MJ (6 mM) and/or peptides at 5 microMolar. Peptide sequences: wt HK1 peptide Ac-MIAAQLLAYYFTELKGRKKRRQRRRPPQ-NH2 (SEQ ID NO:90), CMT4G-mutated HK1 peptide Ac-MGQICQRESATAAEKGRKKRRQRRRPPQ-NH2 (SEQ ID NO:91) and Control peptide Ac-GRKKRRQRRRPPQ-NH2 (SEQ ID NO:92).

FIG. 4. Peptide libraries 1-6 designed for binding optimization to VDAC. 1a and 2a represent the initial sequence of the peptides submitted to the alascan, deletion, optimization and stabilization assays. nL stand for norleucine a non-oxidizable surrogate of methionine. Tat sequence is highlighted in blue while the NHK1 recognition sequence is in red with sequence numbering at the top.

FIG. 5. Time-lapse quantification of mitoGCaMP2 (A) and GCaMP2 (B) fluorescence levels within mitochondria and within the cytosol respectively. Quantifications of fluorescence levels of HEK-293 cells transfected with mitoGCaMP2 (A) and GCaMP2 (B) probes. Control (circle) represents the fluorescence level in mitochondria when treating the cells with the diluents 0.1 DMSO and 5% EtOH used for MJ and compound solubilization. MJ was tested at 6 mM and compound 1a at 33 μM. Statistical analysis using two-way ANOVA followed by Tukeys's multiple comparison tests (N=3 independent experiments). Results are expressed as means±SEM. **p<0.01, ****p<0.0001, ns. non significant. A. U. arbitrary unit

FIG. 6. Effects of alascan (A. B) and deletion (C. D) studies on compounds 1a and 2a. All compounds were tested at 10 μM on the screening assay (N=5 independent experiments). Alascan substitution is bold typed. Statistical analysis showing one-way ANOVA followed by Dunnett's multiple comparison tests between compounds 1a (dark grey plot in A and C) or 2a (dark grey plot in B and D) and the other compounds. Blue plots represent compounds in which alascan studies revealed significant amino acids involved in the interaction with VDAC or compounds in which deletion studies led to a significant loss of activity. *p<0.05, **p<0.01, ***p<0.001. When unspecified, the statistical test is not significant (white plots). Results are expressed as means±SD. A. U, arbitrary unit.

FIG. 7. Effects of the isosteric substitution combinations on the amino acids involved in VDAC interaction in compounds 3c (A) and 4d (B). Substitutions are bold typed. All compounds were tested at 3 μM except compounds 3c and 4d at 10 μM (dark grey plots) and 3 μM (light grey plots) (N=3 independent experiments). Statistical analysis showing one-way ANOVA followed by Dunnett's multiple comparison tests between compounds 3c or 4d at 3 μM and the other compounds. Red plots represent compounds in which isosteric substitution combinations led to the most significant increase of activity. *p<0.05, **p<0.01. When unspecified, the statistical test is not significant. Results are expressed as means±SD. A. U, arbitrary unit.

FIG. 8. A) Structure of the N-terminal modification introduced in 7f. Effect of the introduction of helicogenic Aib (U) in 3c or 5x sequences. Modified amino acids are boldfaced. All compounds were tested at 10 μM (A) and 3 μM (B). Control conditions (dotted line) represent the fluorescence level in mitochondria when treating cells with the diluents 0.1 DMSO and 5% EtOH used for MJ and compound solubilization. MJ (line) represents the fluorescence level in mitochondria when treating the cells with MJ alone at 6 mM. Statistical analysis using one-way ANOVA followed by Dunnett's multiple comparison tests between 3c or 5x (dark grey at 10 μM, light grey at 3 μM) and the other compounds (N=3 independent experiments). Red plots correspond to peptides exhibiting a significant increase of activity. Results are expressed as means±SD. *p<0.05, ***p<0.001. ns. non significant. A. U. arbitrary unit.

FIG. 9: Effects of the SAR study optimizations on mitochondrial Ca²⁺ efflux through VDAC illustrating a gain of activity. Graph shows a representative dose response curve of compounds 1a, 5x and 7g on the screening assay. IC50 are indicated for each compound (N=3 independent experiments). Results are expressed as means±SD. A.U. arbitrary units.

FIG. 10: Study of the stability of NHKI derived sequence (3c′, 7a′, 7d′, 7f′-g′) towards rat serum (N=3 independent experiments). All peptides were tested at a concentration of 66.6 μmol/L in presence of 25% (v/v) of rat serum and water after incubation at 37° C. for 24 h. Errors bars show the standard deviation.

FIG. 11: Effects of NHKI-derived peptides 3c, 5x, 7d and 7g on sciatic nerve explants cultured in medium supplemented with serum. (A) Representative CARS images showing myelin (green) of an intact sciatic nerve collected and immediately fixed in 4% PFA and a sciatic nerve explant cultured in medium supplemented with FBS referred as negative control. (B) Representative CARS images showing myelin (green) of sciatic nerve explants cultured in medium supplemented with FBS containing NHKI-derived peptides at 3 μM for 24 h. All nerves are represented in longitudinal sections. Healthy myelin sheath (white arrows), Node of Ranvier (white stars) and myelin ovoids (orange arrows) are illustrated. Scale bar: 20 μm. (C) Graph showing the percentage of damaged fibers in intact nerves (white plot), negative controls (light grey plot), 3c and its analog 7d (blue plots) 5x and its analog 7g (red plots). (N=3 independent experiments). Results are expressed as means±SD. Statistical analysis using one-way ANOVA followed by Dunnett's multiple comparison tests. *p<0.05, **p<0.01. ns, non-significant.

FIG. 12: AAV9 represents an efficient way to sustain anti-demyelinating peptide expression in target cells. HEK293 cells were infected with a control AAV9 or AAV9-HK peptide or not infected. Two days later cells were incubated with a fluorescent dye Rhod-2 that fluoresces with calcium in mitochondria. 15 minutes later infected cells were incubated with methyl jasmonate (6 mM) and non-infected cells were incubated with methyl jasmonate (6 mM)+5z peptide (5 μM) for 40 minutes. Pictures were taken every 5 minutes imaging Rhod-2 dye.

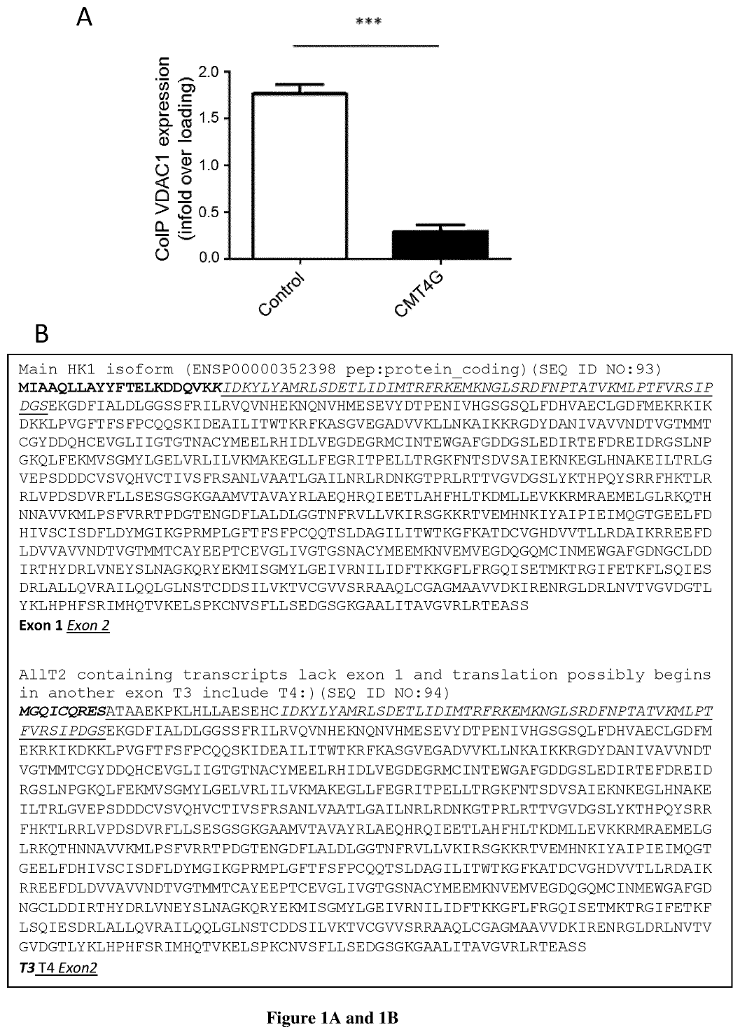

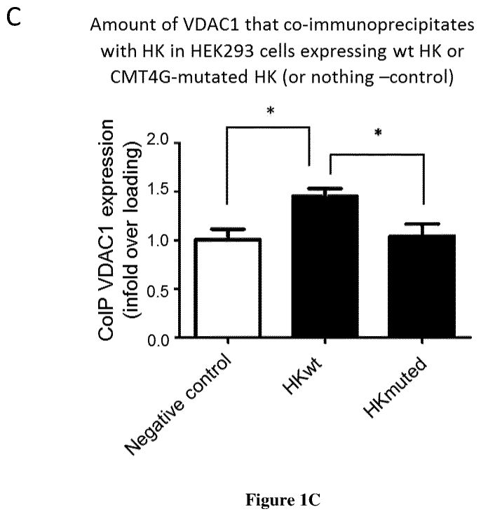

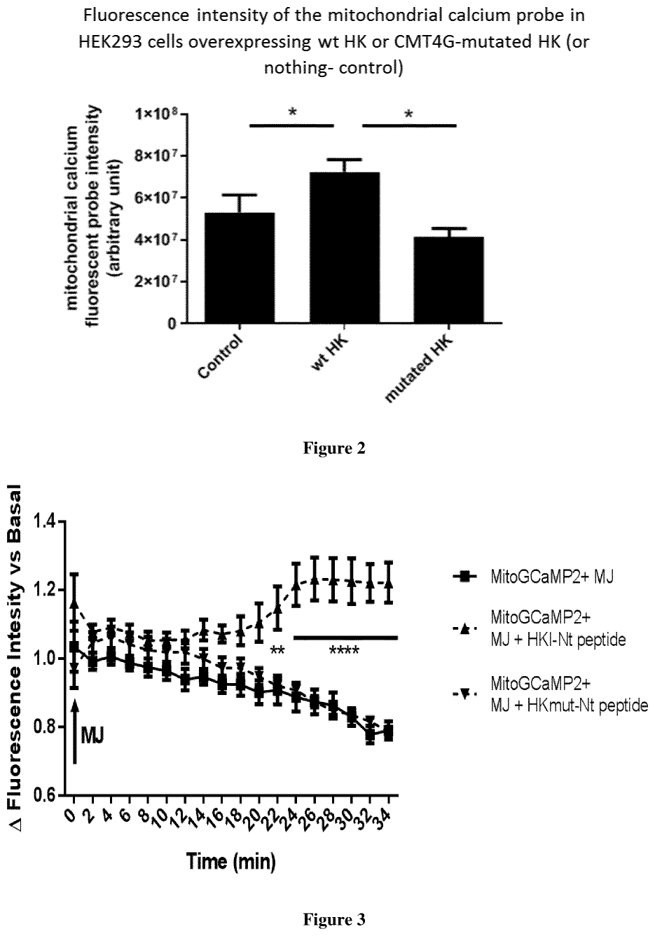

### EXAMPLE 1

### Material & Methods

Peptides 1a-6r used in SAR studies (truncation and Ala-scan) were purchased from Proteomic Solutions (Saint-Marcel, France). Peptides 7a-g, 3c′, 5x′, 7a′-g′ were synthesized on an automated microwave peptide synthesizer CEM Liberty One (CEM Corporation). Amino acids and Rink Amide MBHA resin were purchased from Iris Biotech (Germany), while Rink Amide MBHA LL resin was purchased from Sigma-Aldrich/Novabiochem (St. Louis, MO, USA). Oxyma pure and DIC were acquired from Iris Biotech (Marktredwitz, Germany). HOBt, DIEA, and TIS were obtained from Sigma-Aldrich (St. Louis, MO, USA) while dichloromethane and acetonitrile were obtained from VWR Chemicals (Radnor, Pennsylvania, USA). DMF was obtained from Carlo Erba Reagents (Val de Reuil, France), piperidine from Acros Organics (Illkirch, France) and anhydride acetic from Prolabo (Paris, France). Rat serum and dimethyl sulfoxide were purchased from Sigma-Aldrich (St. Louis, MO, USA). Elastase (from porcine pancreas, EC 3.4.21.36) was purchased from Promega (Madison, WI, USA).

### Solid Phase Peptide Synthesis

All peptides were prepared by standard solid phase peptide synthesis using the Fmoc strategy on a CEM Liberty One microwave-assisted peptide synthesizer. Resins used were Rink Amide MBHA (100-200 mesh, loading 0.67 mmol/g) for the synthesis of 12-16 peptide residues (compounds 3c′, 5x′, 7a′-f′) at 0.1 mmol scale, and Rink Amide MBHA LL (100-200 mesh, loading 0.36 mmol/g) for the 25-29 peptide residues (compounds 5x, 7a-f) at 0.033 mmol or at 0.055 mmol scale. DIC/Oxyma (0.5M/2M in DMF) was used as coupling reagents with a 5-fold excess of each protected aminoacids. In the case of Fmoc-Arg(Pbf)-OH coupling, a double coupling was carried. A 20% piperidine solution in DMF was used for deprotection of the Fmoc group. The resin was swelled in DMF overnight in the reaction vessel, then elongation process was carried out under microwave irradiation (1 mL of DIC+0.5 mL of Oxyma pure at 70° C. (25 W) during 10 min). Deprotection cycles were carried out with a 20% piperidine solution in DMF (7 mL for 30 sec at 75° C., then 7 mL during 3 min at 70° C.). When further modifications/additional aminoacids was needed at N-term part (compounds 7a, 7b, 7d, 7e, 7f), the resin was splitted in 2 or 3. After completion of the synthesis, the peptide-bound resin was washed with 2×15 mL of DMF and with 2×15 mL of DCM. Finally, side chain deprotection and cleavage of the peptide from the resin the peptide was cleaved from the resin by a 2-3 h treatment with TFA/water/triisopropylsilane (95/2.5/2.5). Trifluoacetic acid solution was evaporated under reduced pressure, followed by diethylether precipitation and diethylether washes to afford the crude peptide as a white powder. The analogues were purified by RP-HPLC on a C18-column and identity of the product was established by LCMS. The purity of the peptides was found to be of ≥95% purity for all peptides.

### Analytical HPLC

Peptides were analyzed with a Thermo Fisher Scientific LC-MS device, Accela HPLC coupled to a LCQ Fleet fitted with an electrospray ionization source and a 3D ion-trap analyzer (cone voltage was 30 V). The column used was a Phenomenex BioZen™ 2.6 m Peptide XB-C18 (LC Column 50×2.1 mm), eluting with 0.1% formic acid in water (solvent A) and 0.1% formic acid in acetonitrile (solvent B), using the following elution gradient: 0-2 min, 20% B; 2-5 min, 20-90% B; 5-6 min, 90% B; 7-10 min, 20% B at a flow rate of 0.5 mL/min for a 10 μL injection.

### HPLC Purification

Peptides were purified by semi-preparative HPLC using a Waters 1525 chromatography system fitted with a Waters 2487 tunable absorbance detector set at 214 nm and 254 nm, piloted by Breeze software. A GRACE Vydac C-18 column (250×10 mm, 5 μm) was used, and the flow rate was of 3 mL/min. Two purification gradients were performed depending on the polarity of the peptide.

Method A. The crude peptide was eluted in 0.1% formic acid in water (Buffer A) and in 0.1% formic acid acetonitrile (Buffer B) from A/B (90:10) to A/B (50:50) during 30 min, then A/B (90:10) during 5 min, followed by an isocratic gradient at A/B (90:10) of 2 min.

Method B. The crude peptide was eluted in 0.1% formic acid in water (Buffer A) and in 0.1% formic acid acetonitrile (Buffer B) from A/B (80:20) to A/B (30:70) during 30 min, then A/B (90:10) during 5 min, followed by an isocratic gradient at A/B (90:10) of 2 min.

### CD Spectroscopy

Circular dichroism (CD) experiments were recorded on a Jasco J815 spectropolarimeter. The spectra were obtained in MeOH or in DPBS pH 7 using a 1 mm path length CD cuvette, at 20° C., over a wavelength range of 190-260 nm. Continuous scanning mode was used, with a response of 1.0 s with 0.2 nm steps and a bandwidth of 2 nm. The signal to noise ratio was improved by acquiring each spectrum over an average of three scans. Baseline was corrected by subtracting the background from the sample spectrum. Alpha helical content was determined using the following equation: % Helicity=([θ]obs×100)/(−39500×(1−2.57)/N), where ([θ])obs is the mean residue ellipticity at 220 nm and N the number of peptide bonds.

### NMR Conformational Analysis

NMR samples were prepared by dissolving NHKI analogues (3c′, 7c′, 7d′ and 7g′) in PBS (10% D2O) at pH 6.8 to a final concentration of 2 mM. If required, pH was adjusted using microamounts of 0.1 M NaOH or HCl solutions. In case of solubility issues, up to 10% of DMSO was added. Compounds 3c′, 7d′ and 7f, were studied in presence of 40% TFE (PBS, 10% D20, pH 6.8). Chemical shifts were referenced to trimethylsilylpropanoic acid (TSP).

All spectra were recorded on a Bruker Avance 600 AVANCE III spectrometer equipped with a 5 mm triple-resonance cryoprobe (1H, 13C, 15N) at the “Laboratoire de Mesures Physiques (LMP)” of the University of Montpellier (UM). Homonuclear 2D spectra DQF-COSY, TOCSY (DIPSI2), ROESY, and NOESY were typically recorded in the phase-sensitive mode using the States-TPPI method as data matrices of 256-400 real (t1)×2048 (t2) complex data points; 8-48 scans per t1 increment with 1.0-1.5 s recovery delay and spectral width of 6009 Hz in both dimensions were used. The mixing times were 80 ms for TOCSY and 150 ms for the ROESY/NOESY experiments. Spectra were processed with Topspin (Bruker Biospin) and visualized with Topspin or NMRview 64 on a Linux station. Matrices were zero-filled to 1024 (t1)×2048 (t2) points after apodization by shifted sine-square multiplication and linear prediction in the F1 domain.

### Proteolytic Stability Assay

A stock solution of Elastase at 1 mg/mL was prepared in Tris·HCl buffer (50 mM, pH 8, containing 0.5 mM CaCl₂,). The stock solution was diluted at 0.94 mg/mL with 658 μL of stock solution in 42 μL of Tris·HCl buffer. All peptides were dissolved in DMSO to prepare a 6.66 mmol/L stock solution. A more diluted peptide solution (0.666 mmol/L) was prepared with 70 μL of stock solution in 630 μL of Tris·HCl buffer pH 8. In a 1.5 mL Eppendorf, 890 μL of Tris·HCl pH 8 was introduced followed by 100 μL of peptide solution (0.666 mmol/L) and incubated for 15 min at 37° C. prior to degradation. Then, 10 μL of Elastase solution (0.94 mg/mL) was added. The reaction mixture was incubated up to 4h at 37° C. with shaking at 1000 rpm. Aliquots (50 μL) were taken at different time points, quenched with 450 μL of MeOH, and centrifuged for 20 min (14000 rpm) at 4° C. The supernatant was transferred into an injection vial and analyzed by LC-MS with an eluting program of 0.1% formic acid in water and 0.1% formic acid in acetonitrile (see analytical data section). The relative concentrations of the remaining peptides and the cleavage products were calculated by integration of the corresponding peak in the HPLC chromatogram/MS trace. A control peptide solution was prepared without the enzyme. The hydrolysis of the control peptide solution was found to be stable after 4h at 37° c. in Tris buffer, except for compound 5x′. All proteolytic degradation experiments were carried out in triplicate.

### Structure Calculations

¹H chemical shifts were assigned according to classical procedures. NOE cross-peaks were integrated and assigned within the NMRView software. The volumes of NOE peaks between methylene pair protons were used as reference of 1.8 Å. The lower bound for all restraints was fixed at 1.8 Å and upper bounds at 2.7, 3.3, and 5.0 Å, for strong, medium, and weak correlations, respectively. Pseudo-atom corrections of the upper bounds were applied for unresolved aromatic, methylene, and methyl proton signals as described previously. Structure calculations were performed with AMBER 16 in two stages: cooking, simulated annealing using Generalized Born implicit solvent model. The cooking stage was performed at 1000 K to generate 100 initial random structures. Simulated annealing calculations were carried during 20 ps (20000 steps, 1 fs long). First, the temperature was risen quickly and was maintained at 1000 K for the first 5000 steps, then the system was cooled gradually from 1000 K to 100 K from step 5001 to 18000, and finally, the temperature was brought to 0 K for the 2000 remaining steps. For the 3000 first steps, the force constant of the distance restraints was increased gradually from 2.0 to 20 kcal·mol-1·Å. For the rest of the simulation (step 3001-20 000), the force constant was kept at 20 kcal·mol-1·Å. The 20 lowest-energy structures with no violations >0.3 Å were considered representative of the peptide structure. The representation and quantitative analysis were carried out using MOLMOL and PyMOL

### In-Vitro Metabolic Stability in Rat Serum

Prior to degradation, the protein content of rat serum was determined by Bradford assay and found to be of 108 mg/mL. For each peptide, a stock solution in DMSO was prepared at a 6.66 mmol/L concentration. 70 μL of the solution were taken out and added to 630 μL of MilliQ water to make an aqueous peptide solution (0.666 mmol/L). The reaction consisted in 325 μL of MilliQ water and 125 μL of non-diluted rat serum pre-incubated at 37° C. for about 10-15 min before addition of 50 μL the peptide solution at 0.666 mmol/L. The mixture was incubated at 37° C. with shaking at 1000 rpm. Aliquots (25 μL) were taken at different time points (0 min, 5 min, 15 min, 30 min, 1h, 2h, 3h, 5h, 7h, 24h, 48h) and enzymatic reaction was quenched with 225p L of MeOH to precipitate all serum proteins. The Eppendorf tube was directly centrifuged (14000 rpm) for 20 min at 4° C. to remove precipitated proteins by pelleting. The supernatant was transferred into an injection vial and analyzed by LC-MS with an eluting program of 0.1% formic acid in water and 0.1% formic acid in acetonitrile (see analytical data section). The relative concentrations of the remaining peptides and the cleavage products were calculated by integration of the corresponding peak in the HPLC chromatogram/MS trace.

A control peptide solution was prepared without rat serum. All peptide control solution were found to be stable over 48h in water at 37° C. All serum stability experiments were carried out in triplicate.

### Cell Culture and Transfection

HEK-293 cells were purchased from ATCC (american type culture collection, USA). They were cultured in a humidified incubator at 37° C. with 5% CO2 in DMEM (Gibco, Thermo Fisher Scientific, France) supplemented with 10% heat-inactivated FBS (Gibco, Thermo Fisher Scientific, France) and 1% PS (Gibco, Thermo Fisher Scientific, France).

For the peptide screening assay and live imaging experiments, cells were transfected with mitoGCaMP2 and GCaMP2 plasmids using jet-PRIME reagent (Polyplus-transfection SA, France) according to the manufacturer's recommendations. These two plasmids express the mitochondria-targeting and the cytosolic-targeting GCaMP2 proteins respectively.

### Live Imaging

Live imaging experiments were performed on HEK-293 cells transfected with either the mitoGCaMP2 or the GCaMP2 plasmids. 500,000 cells per well were seeded in 6-well microplate (NUNC, reference 153066, Thermo Fisher Scientific, France) in 1 ml of DMEM supplemented with 10% FBS and 1% PS. 48 h after seeding, cells were transfected with 2 μg of mitoGCaMP2 or GCaMP2 plasmids using jet-PRIME reagent according to the manufacturer's protocol. 48h after transfection, the microplates were placed under a videomicroscope equipped with a humidified chamber at 37° C. and with 5% CO2. Next, 6 mM of pre-heated MJ (37° C.) was added to the wells with or without the peptide 1a at 33 μM in 1 ml of DMEM without red phenol supplemented with 10% FBS and 1% PS, and containing 0.1% DMSO and 5% EtOH. In parallel, wells containing only 1 ml of DMEM without red phenol and with 10% FBS, 1% PS, 0.1% DMSO and 5% EtOH served as control condition. Live imaging acquisition was triggered when adding MJ with or without the peptide 1a. For the control condition, image acquisition was triggered after addition of 1 ml of DMEM without red phenol supplemented with 10% FBS and 1% PS, containing 0.1% DMSO and 5% EtOH. Movies were acquired every 2 min during 30 min using an inverted Zeiss Axio Observer Z1 (Zeiss, France) and a 20×/0.4 objective (Zeiss, France). For each condition, three independent experiments were performed. Overall, 5 ROIs per condition were analyzed using Zen software (Zen 2.3 lite, Zeiss, France) and ImageJ software (version 1.52o, NIH, USA). Results are expressed as the mean±SEM using GraphPad Prism software (version 8.0.1).

### Screening Assay

The activity of the designed compounds was assessed on HEK-293 cells transfected with mitoGCaMP2. 40,000 cells per well were seeded in 96-well-microplates coated with Poly-D-Lysine (reference 655946, Greiner Bio-One, France) in 200 μl of DMEM supplemented with 10% FBS and 1% PS. 24 h after seeding, cells were transfected with 50 ng of mitoGCaMP2 plasmid per well using jet-PRIME reagent (Polyplus-transfection S.A, France) according to the manufacturer's recommendations. 48 h after transfection, a first measure of fluorescence was performed using the microplate reader CLARIOstar® (BMG Labtech, France). This measurement represented the basal level of Ca2+ into the mitochondria upon transfection. After a wash with 100 μl of PBS, cells were incubated with a mixture of pre-heated (37° C.) MJ at a final concentration of 6 mM and compounds at the indicated final concentrations in PBS containing 0.1% DMSO and 5% EtOH. After 35 min in a cell incubator, a second measure of fluorescence was performed using the microplate reader CLARIOstar®. This measure represented the level of mitochondrial Ca2+ according to the peptide activity. Compounds were tested in triplicates per microplates and in three or five independent experiments for each peptide. For dose effect curves, compounds were tested in triplicates per microplates and in three independent experiments. Results are expressed as the ratio between the second and first measures and normalized to the conditions without compounds containing only PBS with 0.1% DMSO and 5% EtOH. Results are expressed as the means±SD in histogram plots and dose response curves using GraphPad Prism software (version 8.0.1).

### Mice Included in the Study

All mouse experiments were approved by the «comité regional d'éthique pour l'expérimentation animale» of Languedoc-Roussillon and the “ministère de la recherche et de l'enseignement supérieur” (authorization 2017032115087316 and 2016091313354892). All the procedures were performed in accordance with the French regulation for the animal procedure (French decrees 2013-118 and 2020-274) and with specific European Union guidelines for the protection of animal welfare (Directive 2010/63/EU). Mice were maintained on a 12 h dark, 12 h light cycle with a humidity between 40 and 60% and an ambient temperature of 21-22° C. Mouse experiments were conducted on twelve-week-old C57BL6/J purchased from Janvier Labs (France).

### Sciatic Nerve Explant Culture and CARS Imaging

Twelve-week-old C57BL6/J mice were euthanized using Pentobarbital (54.7 mg/ml, 100 mg/kg, Centravet, France). First, sciatic nerves were collected, washed in PBS and their epineurium was removed. Next, 5 mm long nerves were put in 24-well microplates (NUNC, Thermo Fisher Scientific, France) in 500 μl of DMEM supplemented with 1% PS and with or without 10% FBS containing the compounds at 3 μM containing 0.1% DMSO, and further incubated in a humidified chamber at 37° C. and 5% CO₂. Negative controls consisted in sciatic nerve explant cultures without compounds (only DMEM supplemented with 1% PS, with or without 10% FBS and 0.1% DMSO). Intact sciatic nerves collected and immediately fixed in 4% PFA served as a control of healthy myelin sheath for CARS imaging. After 24 h in culture, sciatic nerve explants were washed three times with PBS and fixed for 1 h in 4% PFA aqueous solution (Electron Microscopy Sciences, Thermo Fisher Scientific, France) at room temperature. All CARS images were acquired with a two-photon microscope LSM 7 MP coupled to an OPO (Zeiss, France) complemented by a delay line. A ×20 water immersion objective (W Plan Apochromat DIC VIS-IR, Zeiss, France) was used for image acquisition. Each acquisition was conducted in three independent experiments. For each experiment, three ROIs per conditions were used to quantify the percentage of damaged fibers per field using Zen software (Zen 2.3 lite, Zeiss, France). Results are expressed as means±SD.

### Statistical Analysis

Data were analyzed with excel (Microsoft Office Standard 2016) and GraphPad Prism (version 8.0.1) softwares (Graphpad Software) and were expressed as the mean±SD or SEM as indicated in the figure legends. Statistical differences between mean values were tested using one-way ANOVA followed by Dunnett's multiple comparison tests or two-way ANOVA followed by Tukey's multiple comparison tests as indicated in the figure legends. Differences between values were considered significant with: *p<0.05, **p<0.01, ***p<0.001, ****p<0.0001. ns, non-significant.

### Results

### Synthesis of Peptides 1-7.

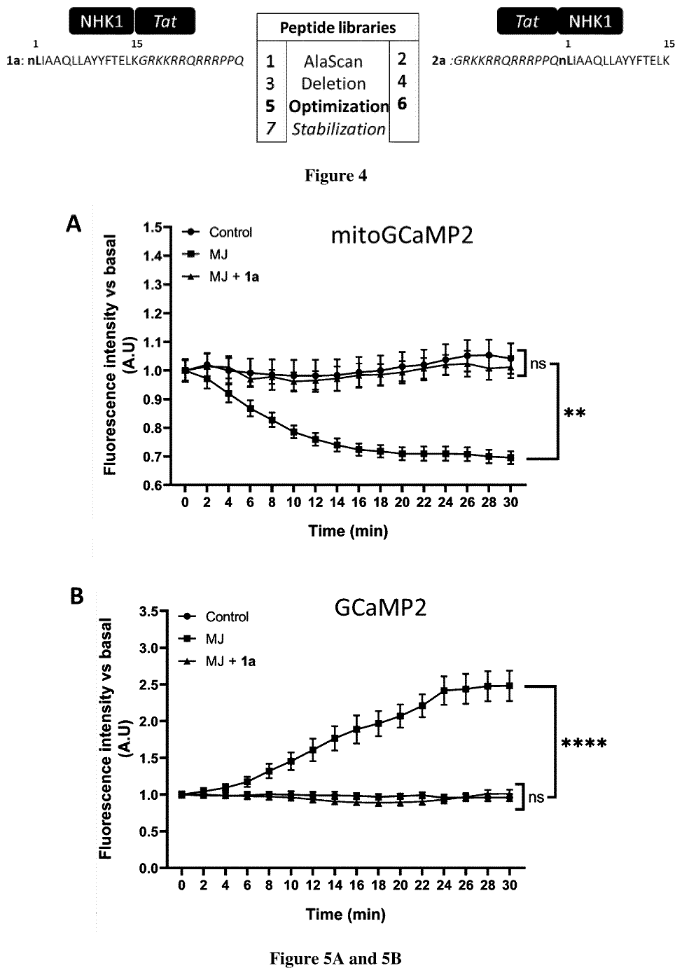

Peptide libraries 1, 2 allowing the alascan, 3, 4 used for the deletion studies and 5 for the first round of optimization were purchased at Proteomic Solutions. Peptide libraries 6 and 7 were synthesized through solid-phase Fmoc/tBu strategy using Rink amide resin. After elongation was completed, the peptides were cleaved from the resin using TFA, affording targeted compounds with yields ranging from x % to x % and a purity of at least 95% for each of the synthetic peptides as judged by HPLC/MS analysis. All peptides 1-7 except the one of library 7′ contains the Tat cell penetrating peptide used to ensure the peptide internalization during the in cellulo-binding assay. Tat was placed on the Cter or Nter of the HK fragment in order to assess its effect on VDAC recognition (FIG. 4).

### Binding Assay.

The biological screening of the peptide is based on the ability of methyl jasmonate (MJ) to binds and detaches HK-1 from mitochondrial VDAC in a time and dose dependent manner.^{16,20} For this purpose, we developed an in-cellulo screening assay in which HEK-293 cells, expressing VDAC and HK,25 were transfected with GCaMP2, a cytoplasmic Ca2+-sensing probe, or with mitoGCaMP2, the same probe addressed to mitochondrial matrix. The use of these probes allowed the monitoring of cytoplasmic and mitochondrial Ca2+ levels in real time as previously shown in vivo. MJ that removes HK from VDAC was used to induce a Ca2+ release outside mitochondria measured through a drop in mitoGCaMP2 fluorescence and an increase in GCaMP2 fluorescence in cells. Compounds mimicking the NHKI sequence can then block this release in presence of MJ and maintain fluorescence levels in mitochondria and cytoplasm through their binding to VDAC. On the other hand, low-activity compounds for VDAC would lead to a fluorescence decrease in mitochondria, and increase in cytoplasm as observed with addition of MJ.

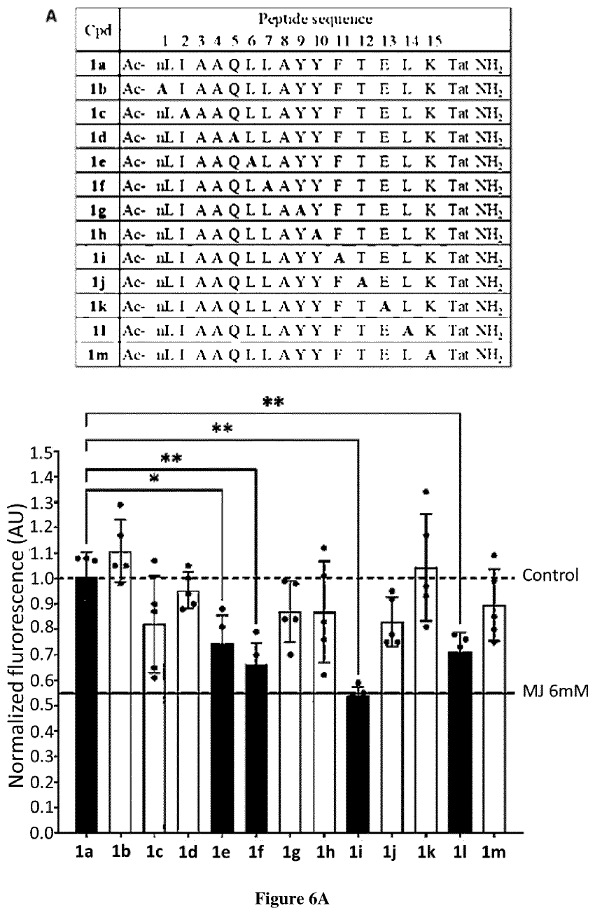

In order to validate this assay, we conducted a timelapse imaging of HEK-293 cells transfected with mitoGCaMP2 or its cytoplasmic form GCaMP2. Basal levels of Ca2+ in mitochondria or in the cytosol remained stable for at least 30 min before treatment. Treatment with MJ (T=0) induced a significant decrease of mitoGCaMP2 fluorescence for at least 30 min (FIG. 5A) indicating a Ca2+ efflux out of mitochondria.

At the opposite and in the same timeframe, GCaMP2 fluorescence increased significantly (FIG. 5B) indicating a cytoplasmic Ca2+ increase concomitant with the mitochondrial Ca2+ release. Any fluorescence change induced by MJ treatment was blocked by peptide 1a mimicking NHK-I sequence (MJ+peptide 1a condition in FIG. 5A) or in the cytosol (MJ+peptide 1a condition in FIG. 5B) indicating that this peptide was able to block mitochondrial calcium release through VDAC along time.

Therefore, this in cellulo system constitutes a relevant assay to measure the activity of compounds on the mitochondrial Ca2+ release through VDAC. Using this assay, the IC50 of compounds 1a and 2a were determined at 15.6±2 μM and 13.9±3.1 μM respectively (data not shown). According to these data, we used these conditions to screen for new peptides activity at 10 μM. The position of the Tat-peptide at the N- or the C-terminus did not influence the activity of the NHKI peptides.

### Alascan on Peptides 1a and 2a

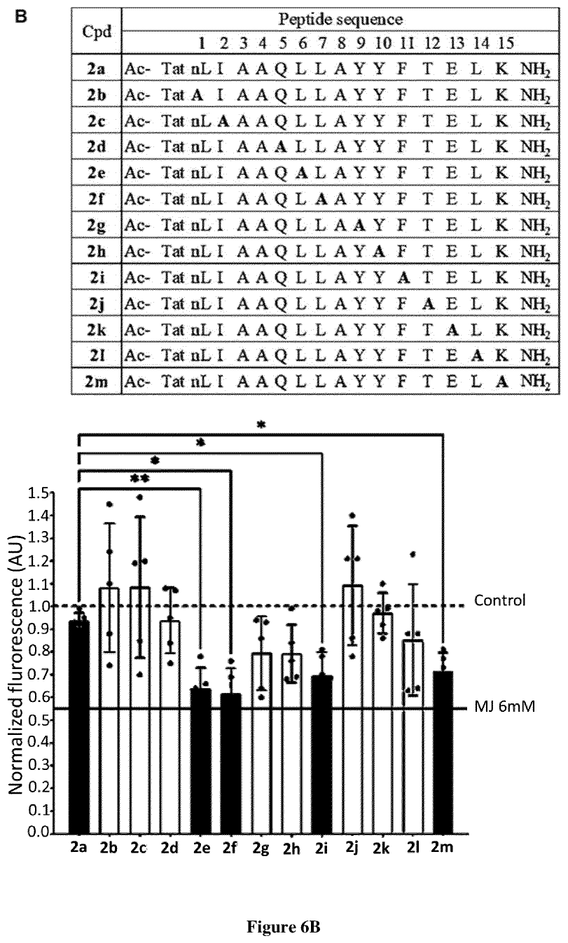

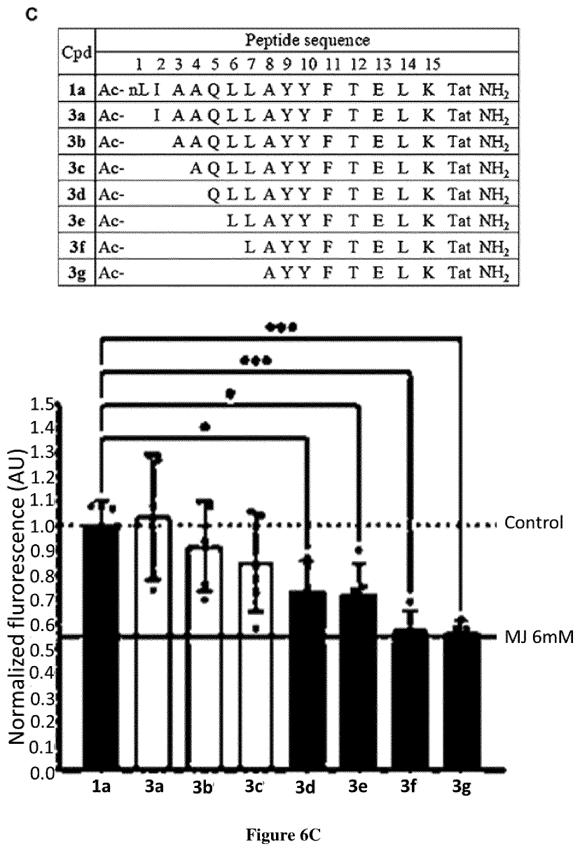

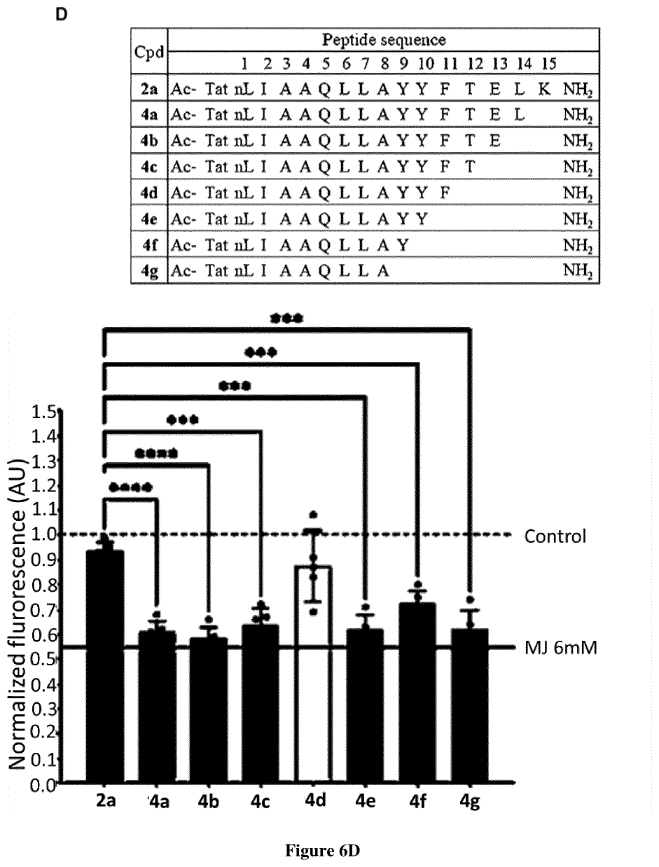

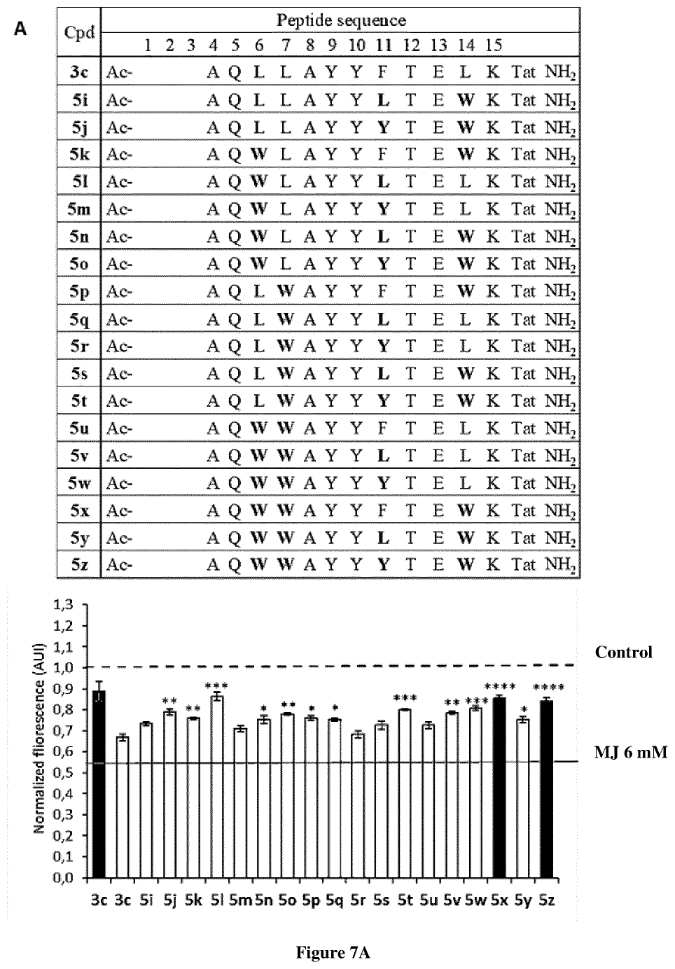

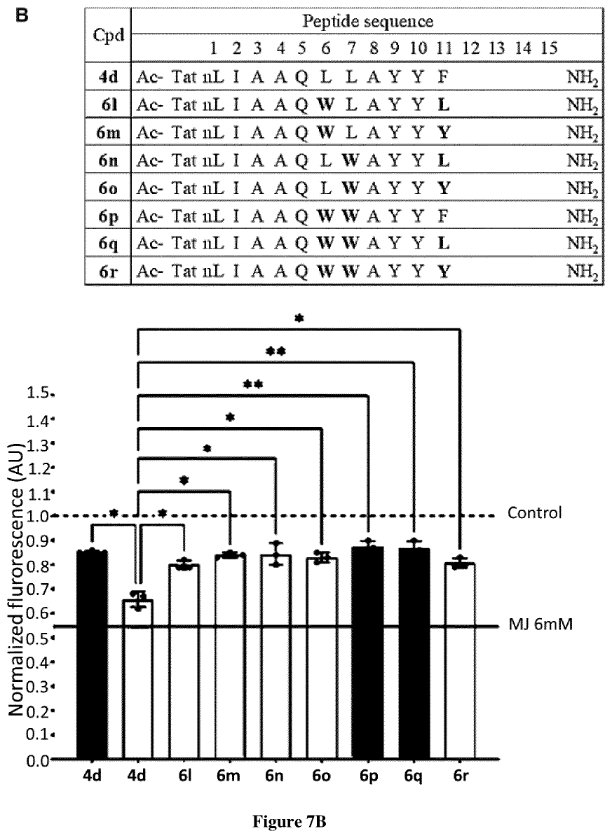

In order to identify the amino acids of peptides 1a and 2a involved in the VDAC recognition we performed an alascan on both peptides. We synthesized twelve derivatives for each peptide in which all amino acids were replaced by an alanine delivering series 1b-m and 2b-m (FIGS. 6A, 6B). The two series that differ by the Tat positioning at the N- or C-terminus for series 1 and 2, respectively were tested at a 10 μM concentration using the binding assay described above. The two series behave in a comparable way. Indeed, the replacements of the leucine 6, 7 and phenylalanine 11 by alanine in NHK1 for compounds 1e-f, 1i, 2e-f and 2i induce a drop in the affinity for VDAC. Substitution of leucine 14 by an alanine in compound 11 induces a similar drop albeit not seen in the corresponding compound 21.

### Deletion Study on Peptides 1a, 2a

This alascan was completed by a deletion study of the NHK1 sequence (FIG. 6C, 6D) in order to identify the minimal sequence useful for a proper binding to VDAC. N-terminal deletion was examined through the synthesis of the seven peptides 3a-g based on 1a and the C-terminal deletion was explored by a comparable set of peptides 4a-g derived from 2a. The two series were tested at a 10 μM concentration. The N-terminal part of NHK1 extending from residues 1 to 3 seems to be non-essential as evidenced by compounds 3a-c. Confirming the alascan results, deletion of leucine 6 and 7 in compounds 3f-g are deleterious for interaction with VDAC. Deletion of the 3 last C-terminal amino acids (ELK) of compound 2a induced a drop of fluorescence as evidenced for compounds 4a-c. Surprisingly, compound 4d retains a significant affinity for VDAC highlighting once again phenylalanine 11 as a key player in the interaction with VDAC. Thus, the hydrophobic sequence AQLLAYYF (SEQ ID NO:89) of the NHK1 peptide constitutes the core of the interaction with VDAC as evidenced by the combination of the alascan and deletion studies.

### Binding Optimization

We thus retained peptides 3c and 4d which sequences were shortened for a second optimization aimed to substitute the amino acids suspected to be involved in the interaction with VDAC by isosteric counterparts. Thus, compound 3c delivers a new series of compounds 5a-h in which the unique threonine in position 12 of the NHK1 sequence was replaced by tyrosine, aspartic acid, asparagine and valine to study the importance of the hydroxyl moiety carried by the threonine. In the same series, leucine 14 was replaced by valine, isoleucine, phenylalanine and tryptophan in order to assess the influence of beta-branched or aromatic amino acids. The same substitutions were applied to leucine 6 and 7 of compound 4d, for which leucine, tryptophan and tyrosine were used as surrogates of phenylalanine 11 (data not shown).

Threonine 12 substitutions in 5a-d were not efficient and even detrimental when the negative charged aspartic acid was introduced (5b). On the contrary, substitution of the leucine by a tryptophan slightly enhanced interaction with VDAC as shown by compounds 5h, and 6d but with a more significant level for 6h in which leucine 7 was replaced. Finally, substitution of leucine 14 by a tryptophan in compound 5h also significantly led to a gain of activity. Thus, NHK1 interactions with VDAC are mostly mediated by hydrophobic residues, and tryptophan considered as the most hydrophobic residue accordingly to amino acid hydrophobicity scale reinforces such interaction.^{35,36}

To capitalize on these results, the modifications with positive effects were combined to deliver compounds 5i-z and 6l-r tested at 3 μM and compared with 3c and 4d, respectively (FIG. 6). For series of compounds 5i-z and 6l-r, compounds 3c and 4d serve as benchmark. Reducing the concentration of reference from 10 μM to 3 μM allow to maintain VDAC in a partial closed state that better differentiates compounds blocking the calcium efflux. While individual substitutions of hydrophobic leucine 6 or 7 by a tryptophan were accompanied by a moderate affinity increase, combining the two substitutions in a single peptide was more significant as shown by compounds 5x, 5z and 6q. Nevertheless, substitution of phenylalanine 11 is less obvious to analyze but leucine in 5l, 6q or tyrosine in 5t, 5m, 6m, 6o are equally accommodated at this position. Finally, substitution of leucine 14 by a tryptophan has also a beneficial effect for compounds 5l, 5t, 5x and 5z.

### Binding Optimization Through Reinforcement of Helical Folding

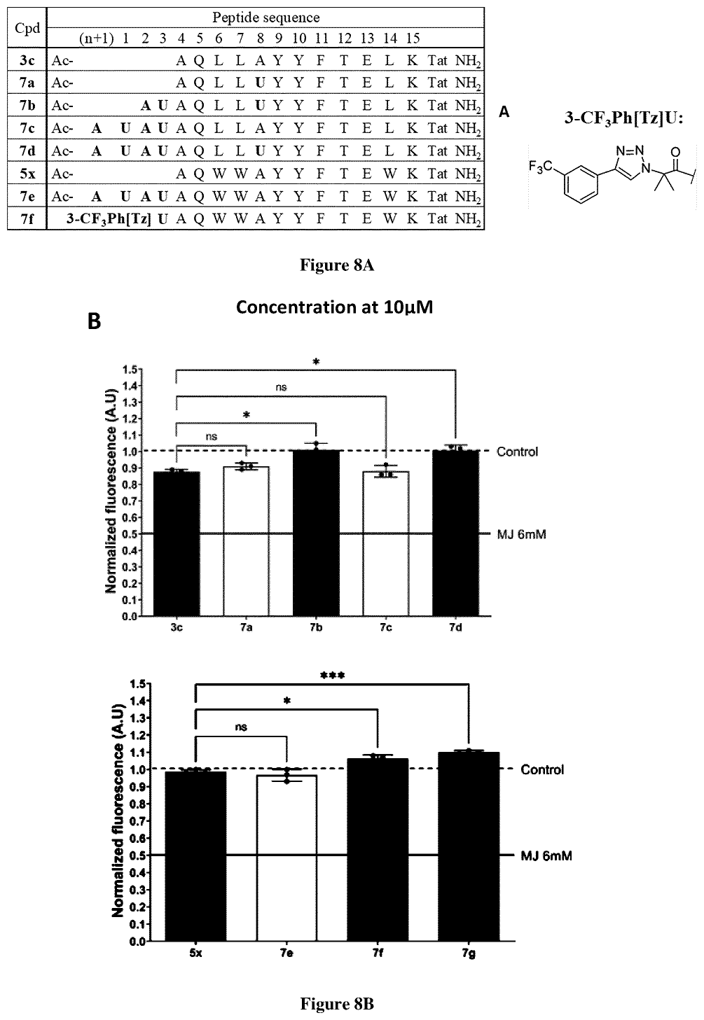

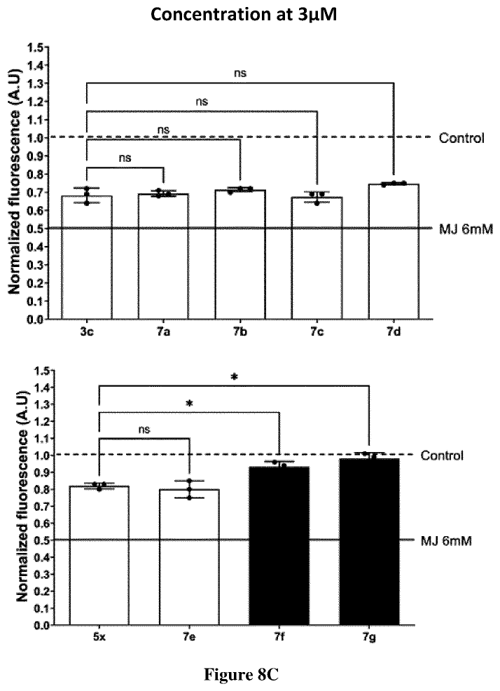

The last modification we introduced, was aimed to take into account the helical fold adopted by the NHK1 sequence³⁷. For this purpose, a sequence alternating alanine and a-aminoisobutyric acid (Aib, U) both of which are a-helix inducers was introduced at the N-terminal side of the HK-1 sequence delivering peptides series 7. Furthermore, this modification was expected to reduce the susceptibility of the compounds towards proteolytic cleavage. Additionally, as hydrophobic interactions conditioned proper VDAC interactions, we introduced the 3-CF₃Ph[Tz]U dipeptide as N-terminal capping that was shown in a previous study to enhance peptide insertion within membrane (FIG. 8A).³⁸

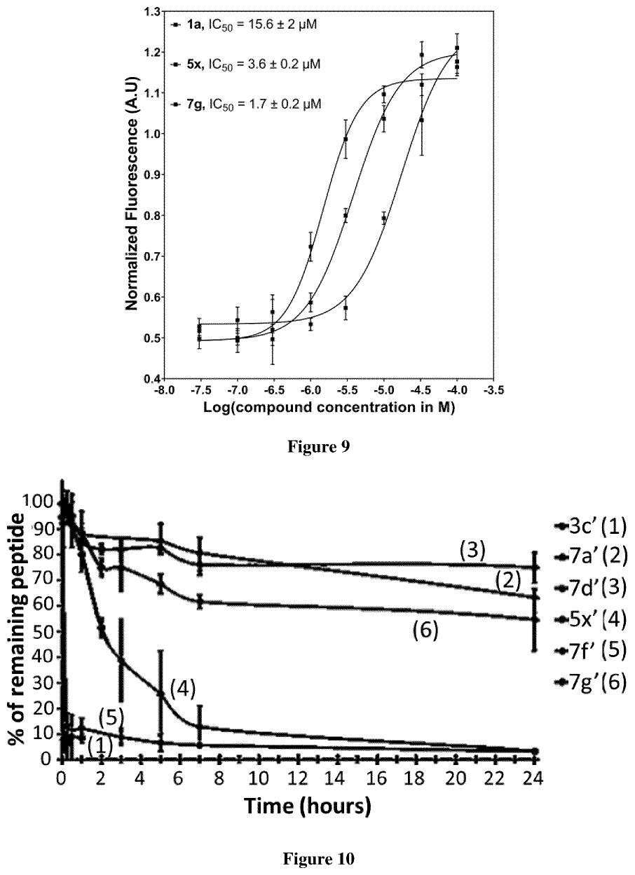

Series 7a-g was tested at 10 and 3 μM on the screening assay (FIG. 8A-B). Among this series, while 7b, 7d, 7f and 7g were significantly more active than compounds 3c and 5x (FIG. 8A), when tested at 10 μM, only 7f and 7g still exhibited a significant higher activity at 3 μM (FIG. 8B). Consequently, in order to precisely define their activity, 7f and 7g were tested in a dose response manner on the screening assay (data not shown). Their IC₅₀ were evaluated at 2.6±0.6 μM and 1.7±0.2 μM respectively. To summarize this SAR study, the AQLLAYYF sequence (SEQ ID:36) of HK contains the critical residues involved in the interaction with HK. More specifically, the hydrophobic patch constituted by leucine 6, 7 and phenylalanine 11 governs the interaction and their replacement by tryptophan enhanced the interaction. Moreover, the different steps of optimizations lead to a 10-fold increase in activity on the mitochondrial Ca²′ efflux through VDAC as shown by the dose response experiments performed for compounds 1a, 5x and 7g on the screening assay (FIG. 9). Furthermore, helical wheel projection of the different series of compounds placed the residues involved in VDAC interaction on the same face of the helix (data not shown).

### Circular Dichroism

Compounds 3c, 5x, 7a-f in PBS buffer were analyzed by circular dichroism. While compounds 3c, 5x, 7a are not structured, compounds 7b-f present negative maxima around 210 and 220 nm compatible with a peptide partially structured in a-helix (data not shown). In order to assess the contribution of the Aib introduced on the N-terminal in the structuration of the NHK1 peptide, compounds 3c′, 7a′, 7b′ and 7c′, without Tat sequence, were synthesized (data not shown). In PBS solution, the AU repeated sequence introduced at the N-terminus of the NHK sequence allow the compound to fold gradually as a helix as observed for compounds 7b′ and 7c′ (data not shown). As in PBS solution the CD spectra cannot extend on the whole wavelength range of interest, we performed the analysis in methanol. While adopting a random coil structure in PBS, compound 3c begins to fold as an helix due to the kosmotropic effect of methanol. Nevertheless, the tendency observed in PBS for a folding depending of the amount of Aib introduced in the sequence was confirmed as compound 7d′ containing three Aib is the most structured of the series (data not shown).

### Proteolytic Stability Assay

Although Tat was shown to be an appropriate CPP for the delivery of bioactive cargos such as the NHK1 derived peptides, its use is tempered by a poor serum stability.^{39,40} Indeed, the Tat sequence half-life in serum is less than 6 min.^{41,42} Furthermore, serum is constituted by a blend of enzyme that do not allow to identify the different cleavage sites. In a preliminary experiment, a solution of peptide 3c in 25% rat serum confirm this instability as only 10% of the 3c remains after 5 min (data not shown). Moreover, multiple cleavage sites produce too many fragments whose concentration are under the detection limits of the LC-MS apparatus precluding their identification. Therefore, it is generally more convenient to use a defined proteolytic enzyme to identify the preferred cleavage sites.

Among the enzyme available in our laboratory the serine endopeptidase elastase (EC 3.4.21.36), found in pancreas as in blood serum, was selected for a marked primary specificity towards alanine and leucine at P1 position, two amino acids that are present in the AQLLAYYF sequence (SEQ ID NO:89) which need to remains intact for VDAC recognition, but are absent in TAT.^{43,44} Therefore, as the NHK1 sequence is crucial for a proper binding to VDAC we focused our effort on the study of the NHK1 sequence without Tat and exposed compounds 3c′, 5x′, 7a′ and 7d′-f to elastase over a period of two hours in Tris·HCl buffer at pH 8 (data not shown).

From this set of compounds, peptides 3c′ and 5x′ containing the NHKI sequence that induced the highest activity after the first optimization step, were fully degraded in less than 30 minutes. Adding the AUAU patch at the N-terminus of these peptides in 7c′ and 7e′ was ineffective to improve their metabolic stability (data not shown). However, the alanine substitution in position 8 by an Aib for 7a′ and 7d′ improve their stability towards elastase. In addition, compound 7f in which the N-terminus was capped with a triazole derivative was the most stable compound despite the fact that the alanine in position 8 was conserved (data not shown). It is noteworthy that the main cleavage site for elastase was at the C-terminus of the alanine 8 since the compounds with the shortened half-life (3c′, 5x′, 7c′ and 7e′) contained this alanine. Thus, enzymatic fragments were in accordance with elastase specificity and replacing alanine in position 8 by an Aib improved stability.

In order to verify whether this stabilization was maintained in more complex media, compounds 7a′, 7d′ and 7f exhibiting the highest stability towards elastase and compound 7g′, an analog of 7f bearing an Aib at position 8 instead of alanine were tested in rat serum which contains hundreds of peptidases.43 Compound 3c′ and 5x′ served as a reference (FIG. 10).

In accordance with the data obtained with elastase, compound 3c′ was readily processed by the proteolytic enzymes present in rat serum, and only 8% remained after 1h (FIG. 10). Compound 7f′ disappeared at a comparable rate, suggesting that simply capping the peptide with a triazole group was not sufficient in serum. However, replacing the alanine in position 8 by an Aib in 7g′ maintained 54% of the compound after 24h. Compound 7a′ showed slower degradation over time, with about 65% of remaining compound after 24 h incubation, while 7d′, analog of 7a′ containing the AUAU patch in N-terminus, exhibited the best serum peptidases resistance with 75% of remaining compound (FIG. 10). The enzymatic fragments were identified by high resolution tandem mass spectrometry.

To conclude, these stability studies showed that alanine at position 8 appears to be a preferential site for enzymatic cleavage of NHKI-derived peptides. Indeed, addition of Aib at position 8 enhanced the NHKI stability towards serum proteases. This stability was further reinforced by capping the N-terminal with 3-CF3-Ph[Tz]U derivative or the AUAU patch.

### Ex Vivo Activity on Sciatic Nerve Explant Cultures

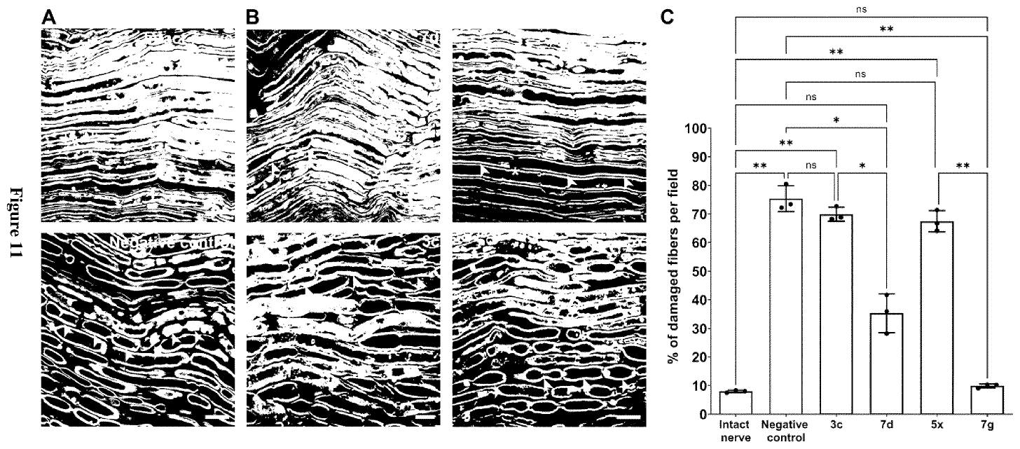

Next, we tested NHKI-derived compound activity on sciatic nerve explant cultures in which Schwann cells demyelinate through a mechanism involving mitochondrial Ca2+ release through VDAC1.44,45,9 Intact myelin was imaged and quantified in sciatic nerve explants using Coherent Anti-Stokes Raman Scattering (CARS) nonlinear microscopy. This imaging method does not require any specific labeling and is suitable for myelin sheath analysis.47,48 In an intact sciatic nerve imaged using CARS, the myelin sheath produced by SC forms a continuous line surrounding the axons (FIG. 11), except at the nodes of Ranvier (FIG. 11). 24 h after incubating nerves in cell culture medium, a spontaneous demyelination occurs which is characterized by formation of ovoids (FIG. 11). Demyelination was quantified by measuring the percentage of damaged fibers, i.e. displaying ovoid formation, over the total number of fibers imaged.

In a first set of experiments, the most active compounds of the screening assay, ie 7d and 7g, and the related compounds 3c and 5x used as control, were tested at 3 μM without serum in the culture medium (data not shown). After 24h in serum-free medium, while compound 3c exhibited the same percentage of damaged fibers as the negative control, all the other compounds significantly reduced the level of damaged fibers (data not shown). In agreement with our previous experiments, the optimized compounds 7d and 7g were significantly more active than the reference compounds 3c and 5x. Moreover, these optimized compounds notably exhibited the same myelin sheath pattern as an intact nerve (data not shown). The results obtained in serum-free medium conditions correlate with those resulting from the screening assay since the optimized compounds have an enhanced activity on the blocking of mitochondrial Ca2+ release.

In the next set of experiments, the same compounds were tested at 3 μM in medium supplemented with serum (FIG. 11A-C). Among all the tested compounds, only treatments with compounds 7d and 7g significantly decreased nerve fibers damage indicating that these peptides were both effective to block demyelination and stable long enough in serum to be effective. Notably, these two compounds were able to significantly preserve the myelin sheath at a similar level to an intact nerve (FIG. 11A-C).

To conclude the results confirmed the higher proteolytic stability of compounds 7d and 7g and, thus the positive effect of the A8U substitution in position 8 used in combination with the AUAU patch or the triazole moiety at N-terminus.

### DISCUSSION

Two molecular models based on the structures of VDAC1 bound to HK1 and HK2 were proposed^{46,47}. Both models have a similar shape with HK located at the pore top thus closing the channel. The 25 residues constituting the HK N-terminal helix are wedged between the N-terminal VDAC helix and the wall of the barrel. Mutation of serine in HK2 to the apolar leucine increased the mutant stability and its binding to VDAC. An Ala-scan combined with a deletion study allow us to identify the AQLLAYYF sequence (SEQ ID NO:89) and its leucine and phenylalanine as pivotal in the VDAC interaction. Substitution of these three amino acids by more hydrophobic ones such as tryptophan reinforced the interaction with VDAC, thus confirming the hypothesis of a relation between hydrophobicity and binding capacity.⁴⁸ The N-terminal sequence of HK adopting an helical fold³⁷ we considered the possibility that the leucine 6, 7 and the phenylalanine 11 constitute an hydrophobic patch located on the same face of the helix. Therefore, we tried to reinforce the helical fold by adding helix inducer such as Aib in the HK sequence. As expected, such substitutions induce a helical fold that was more pronounced in organic solvent like methanol than in buffer solution and was accompanied with an increased binding affinity to the pore. Nevertheless, this result was counterintuitive with respect of the molecular model that plugged the N-terminal helix within the water filed pore. Indeed, while the porin channel is mostly positively charged, the negative charged residues E66, E73, K74, D78, E189, E203, located on the cytoplasm exposed loops of VDAC have been identified to be essential in the binding of HK⁴⁹, little is known about the residues of the N-terminal region of HK participating to the binding.⁵⁰ Indeed, the N-terminal helix of HK essential to a proper interaction with VDAC is mostly constituted of hydrophobic residues and thus are unable to directly bind to the charged VDAC residues. Nevertheless, different studies highlight E73 as a key residue for HK binding⁵¹ and this is supported by the E73Q mutation that abolishes HK1 binding. E73 has an unusual location at the outer face of the b-barrel and point toward the membrane^{52,3,4}. E73 was also identified by photo-affinity approaches as privileged binding site for cholesterol and neurosteroids⁵³. In this case, steroid binding to VDAC do not affect its conductance capacity but more likely suggest that the steroid binding sites are implicated in channel dimerization or hexokinase-mediated signaling. Evidence that cholesterol loading affect HK binding to VDAC has led to the development of olesoxime, a cholesterol hydroxamate derivative. It was recently shown that the highly hydrophobic olesoxime does not enter the water filled VDAC pore but instead interacts at the protein lipid interface⁵⁴. Thus, compound 7f with the hydrophobic 3-CF₃—Ar[Tz] tag might behave in a comparable way interacting with the hydrophobic exterior of the VDAC's b-barrel as this was also suggested for the HK helical helix that is supposed to be inserted in the lipid bilayer⁵⁰. Furthermore, different small molecules characterized by a similar molecular pattern as the one present in compound 7f are able to interact with VDAC-1 and their binding was determined by microscale thermophoresis^{55,56}. The nature of the hydrophobic stabilized helix we developed in this work prompt us to favor a direct interaction of the helix at the membrane interface between the membrane and VDAC. Thus, as recently proposed in a model sustained by electrophysiological measurement the HK helix can be defined as a membrane anchor initiating the HK/VDAC interaction.⁵⁷ In this context, such helix can serve as tools for the development of crosslinking probes able to correctly place the NHK1 sequence on VDAC interface.

### EXAMPLE 2

We produced an AAV9 virus expressing HK peptide 5z (AAV9-HK peptide). HEK293 cells were infected with a control AAV9 or AAV9-HK peptide or not infected. Two days later cells were incubated with a fluorescent dye Rhod-2 that fluoresces with calcium in mitochondria. 15 minutes later infected cells were incubated with methyl jasmonate (6 mM) and non-infected cells were incubated with methyl jasmonate (6 mM)+5z peptide (5 μM) for 40 minutes. Pictures were taken every 5 minutes imaging Rhod-2 dye.

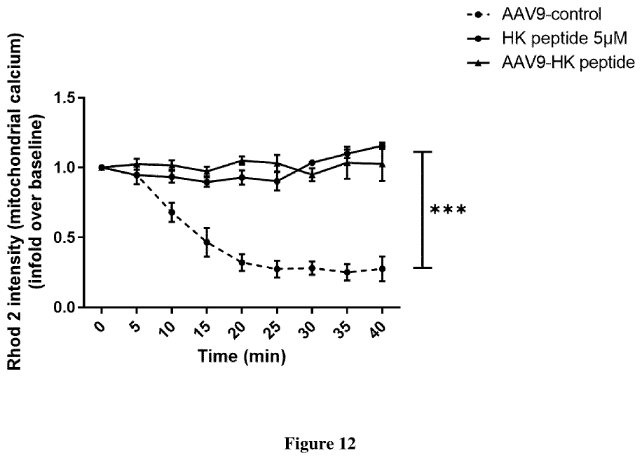

Methyl jasmonate induced a decrease of Rhod-2 fluorescence in mitochondria of cells infected with control virus similar to the decrease seen in non-infected cells in previous experiments. In cells infected with the virus expressing 5z peptide or in cells treated with 5z peptide no decrease occurred (FIG. 12)

This indicates that AAV9 virus expressing 5z peptide prevents mitochondrial calcium release in presence of methyl jasmonate such as peptide 5z does. AAV9 expression represents an efficient way to sustain anti-demyelinating peptide expression in target cells.

### REFERENCES

Throughout this application, various references describe the state of the art to which this invention pertains. The disclosures of these references are hereby incorporated by reference into the present disclosure.
1. Sander, P., Gudermann, T. & Schredelseker, J. A Calcium Guard in the Outer Membrane: Is VDAC a Regulated Gatekeeper of Mitochondrial Calcium Uptake? Int. J. Mol. Sci. 22, (2021).
2. Shoshan-Barmatz, V. et al. VDAC, a multi-functional mitochondrial protein regulating cell life and death. Mol. Aspects Med. 31, 227-285 (2010).
3. Hiller, S. et al. Solution Structure of the Integral Human Membrane Protein VDAC-1 in Detergent Micelles. Science 321, 1206-1210 (2008).
4. Ujwal, R. et al. The crystal structure of mouse VDAC1 at 2.3 Å resolution reveals mechanistic insights into metabolite gating. Proc. Natl. Acad. Sci. U.S.A 105, 17742-17747 (2008).
5. Choudhary, O. P. et al. Structure guided simulations illuminate the mechanism of ATP transport through VDAC1. Nat. Struct. Mol. Biol. 21, 626-632 (2014).
6. De Pinto, V. et al. Determination of the Conformation of the Human VDAC1 N-Terminal Peptide, a Protein Moiety Essential for the Functional Properties of the Pore. ChemBioChem 8, 744-756 (2007).
7. Manzo, G. et al. Folded Structure and Membrane Affinity of the N-Terminal Domain of the Three Human Isoforms of the Mitochondrial Voltage-Dependent Anion-Selective Channel. ACS Omega 3, 11415-11425 (2018).
8. Shoshan-Barmatz, V., Pittala, S. & Mizrachi, D. VDAC1 and the TSPO: Expression, Interactions, and Associated Functions in Health and Disease States. Int. J. Mol. Sci. 20, 3348 (2019).
9. Winquist, R. J. & Gribkoff, V. K. Targeting putative components of the mitochondrial permeability transition pore for novel therapeutics. Biochem. Pharmacol. 177, 113995 (2020).
10. Klapper-Goldstein, H. et al. VDAC1 in the diseased myocardium and the effect of VDAC1-interacting compound on atrial fibrosis induced by hyperaldosteronism. Sci. Rep. 10, (2020).
11. Wilting, F. et al. The antiarrhythmic compound efsevin directly modulates voltage-dependent anion channel 2 by binding to its inner wall and enhancing mitochondrial Ca2+ uptake. Br. J. Pharmacol. 177, 2947-2958 (2020).
12. van Delft, M. F. et al. A small molecule interacts with VDAC2 to block mouse BAK-driven apoptosis. Nat. Chem. Biol. 15, 1057-1066 (2019).
13. Shoshan-Barmatz, V., Krelin, Y., Shteinfer-Kuzmine, A. & Arif, T. Voltage-Dependent Anion Channel 1 As an Emerging Drug Target for Novel Anti-Cancer Therapeutics. Front. Oncol. 7, (2017).
14. Zhang, E. et al. Preserving Insulin Secretion in Diabetes by Inhibiting VDAC1 Overexpression and Surface Translocation in β Cells. Cell Metab. (2018) doi:10.1016/j.cmet.2018.09.008.
15. Pittala, S. et al. The VDAC1-based R-Tf-D-LP4 Peptide as a Potential Treatment for Diabetes Mellitus. Cells 9, (2020).
16. Sohlang, M. N. & Majaw, S. Altered VDAC-HK association and apoptosis in mouse peripheral blood lymphocytes exposed to diabetic condition: an in vitro and in vivo study. Arch. Physiol. Biochem. 1-11 (2021) doi:10.1080/13813455.2020.1867187.
17. Kim, J. et al. VDAC oligomers form mitochondrial pores to release mtDNA fragments and promote lupus-like disease. Science 366, 1531-1536 (2019).
18. Smilansky, A. et al. The Voltage-dependent Anion Channel 1 Mediates Amyloid R Toxicity and Represents a Potential Target for Alzheimer Disease Therapy. J. Biol. Chem. 290, 30670-30683 (2015).
19. Shoshan-Barmatz, V., Nahon-Crystal, E., Shteinfer-Kuzmine, A. & Gupta, R. VDAC1, mitochondrial dysfunction, and Alzheimer's disease. Pharmacol. Res. 131, 87-101 (2018).
20. Rostovtseva, T. K. et al. α-Synuclein Shows High Affinity Interaction with Voltage-dependent Anion Channel, Suggesting Mechanisms of Mitochondrial Regulation and Toxicity in Parkinson Disease*. J. Biol. Chem. 290, 18467-18477 (2015).
21. Karachitos, A., Grobys, D., Kulczynska, K., Sobusiak, A. & Kmita, H. The Association of VDAC with Cell Viability of PC12 Model of Huntington's Disease. Front. Oncol. 6, (2016).
22. Israelson, A. et al. Misfolded Mutant SOD1 Directly Inhibits VDAC1 Conductance in a Mouse Model of Inherited ALS. Neuron 67, 575-587 (2010).
23. Magri, A. et al. Hexokinase I N-terminal based peptide prevents the VDAC1-SOD1 G93A interaction and re-establishes ALS cell viability. Sci. Rep. 6, 34802 (2016).
24. Pittala, S., Krelin, Y., Kuperman, Y. & Shoshan-Barmatz, V. A Mitochondrial VDAC1-Based Peptide Greatly Suppresses Steatosis and NASH-Associated Pathologies in a Mouse Model. Mol. Ther. 27, 1848-1862 (2019).
25. Pittala, S., Krelin, Y. & Shoshan-Barmatz, V. Targeting Liver Cancer and Associated Pathologies in Mice with a Mitochondrial VDAC1-Based Peptide. Neoplasia N. Y. N 20, 594-609 (2018).
26. Bryan, N. & Raisch, K. P. Identification of a mitochondrial-binding site on the N-terminal end of hexokinase II. Biosci. Rep. 35, (2015).
27. Abramov, A. Y., Berezhnov, A. V., Fedotova, E. I., Zinchenko, V. P. & Dolgacheva, L. P. Interaction of misfolded proteins and mitochondria in neurodegenerative disorders. Biochem. Soc. Trans. 45, 1025-1033 (2017).
28. Magri, A. & Messina, A. Interactions of vdac with proteins involved in neurodegenerative aggregation: an opportunity for advancement on therapeuti c molecules. Curr. Med. Chem. 24, 4470-4487 (2017).
29. Tricaud, N. et al. Schwann cell demyelination is triggered by a transient mitochondrial calcium release through Voltage Dependent Anion Channel 1. bioRxiv 581157 (2019) doi:10.1101/581157.
30. Tricaud, N. & Park, H. T. Wallerian demyelination: chronicle of a cellular cataclysm. Cell. Mol. Life Sci. 74, 4049-4057 (2017).
31. Goldin, N. et al. Methyl jasmonate binds to and detaches mitochondria-bound hexokinase. Oncogene 27, 4636-4643 (2008).
32. Magalon, K. et al. Olesoxime accelerates myelination and promotes repair in models of demyelination. Ann. Neurol. 71, 213-226 (2012).
33. Weber, J. J., Clemensson, L. E., Schiöth, H. B. & Nguyen, H. P. Olesoxime in neurodegenerative diseases: Scrutinising a promising drug candidate. Biochem. Pharmacol. 168, 305-318 (2019).
34. Hantke, J. et al. A mutation in an alternative untranslated exon of hexokinase 1 associated with hereditary motor and sensory neuropathy—Russe (HMSNR). Eur. J. Hum. Genet. EJHG 17, 1606-1614 (2009).
35. Wilce, M. C. J., Aguilar, M.-Isabel. & Hearn, M. T. W. Physicochemical Basis of Amino Acid Hydrophobicity Scales: Evaluation of Four New Scales of Amino Acid Hydrophobicity Coefficients Derived from RP-HPLC of Peptides. Anal. Chem. 67, 1210-1219 (1995).
36. Wimley, W. C. & White, S. H. Experimentally determined hydrophobicity scale for proteins at membrane interfaces. Nat. Struct. Biol. 3, 842-848 (1996).
37. Mulichak, A. M., Wilson, J. E., Padmanabhan, K. & Garavito, R. M. The structure of mammalian hexokinase-1. Nat. Struct. Biol. 5, 555-560 (1998).
38. Das, S. et al. Enhancing the Antimicrobial Activity of Alamethicin F50/5 by Incorporating N-terminal Hydrophobic Triazole Substituents. Chem.—Eur. J. 23, 17964-17972 (2017).
39. Koren, E., Apte, A., Sawant, R. R., Grunwald, J. & Torchilin, V. P. Cell-penetrating TAT peptide in drug delivery systems: Proteolytic stability requirements. Drug Deliv. 18, 377-384 (2011).
40. Young Kim, H., Young Yum, S., Jang, G. & Ahn, D.-R. Discovery of a non-cationic cell penetrating peptide derived from membrane-interacting human proteins and its potential as a protein delivery carrier. Sci. Rep. 5, 11719 (2015).
41. Stalmans, S. et al. Cell-Penetrating Peptides Selectively Cross the Blood-Brain Barrier In Vivo. PLOS ONE 10, e0139652 (2015).
42. Rizzuti, M., Nizzardo, M., Zanetta, C., Ramirez, A. & Corti, S. Therapeutic applications of the cell-penetrating HIV-1 Tat peptide. Drug Discov. Today 20, 76-85 (2015).
43. Lee, V. H. L. & Yamamoto, A. Penetration and enzymatic barriers to peptide and protein absorption. Adv. Drug Deliv. Rev. 4, 171-207 (1989).
44. Hooper, N. M. Proteolytic enzymes: A practical approach Edited by R J Beynon and J S Bond. pp 259. IRL Press at Oxford University Press, Oxford. 1989. £29 (spiral bound)/£19 (paper) ISBN 0-19-963058-5/963059-3. Biochem. Educ. 18, 55-55 (1990).
45. Maffioli, E. et al. High-Resolution Mass Spectrometry-Based Approaches for the Detection and Quantification of Peptidase Activity in Plasma. Molecules 25, 4071 (2020).
46. Rosano, C. Molecular model of hexokinase binding to the outer mitochondrial membrane porin (VDAC1): Implication for the design of new cancer therapies. Mitochondrion 11, 513-519 (2011).
47. Zhang, D., Yip, Y. M. & Li, L. In silico construction of HK2-VDAC1 complex and investigating the HK2 binding-induced molecular gating mechanism of VDAC1. Mitochondrion 30, 222-228 (2016).
48. Gelb, B. D. et al. Targeting of hexokinase 1 to liver and hepatoma mitochondria. Proc. Natl. Acad. Sci. 89, 202-206 (1992).
49. Abu-Hamad, S., Zaid, H., Israelson, A., Nahon, E. & Shoshan-Barmatz, V. Hexokinase-I Protection against Apoptotic Cell Death Is Mediated via Interaction with the Voltage-dependent Anion Channel-1: MAPPING THE SITE OF BINDING. J. Biol. Chem. 283, 13482-13490 (2008).
50. Xie, G. & Wilson, J. E. Rat brain hexokinase: The hydrophobie N-terminus of the mitochondrially bound enzyme is inserted in the lipid bilayer. Arch. Biochem. Biophys. 267, 803-810 (1988).
51. Zaid, H., Abu-Hamad, S., Israelson, A., Nathan, I. & Shoshan-Barmatz, V. The voltage-dependent anion channel-1 modulates apoptotic cell death. Cell Death Differ. 12, 751 (2005).
52. Bayrhuber, M. et al. Structure of the human voltage-dependent anion channel. Proc. Natl. Acad. Sci. U.S.A 105, 15370-15375 (2008).
53. Cheng, W. W. L. et al. Multiple neurosteroid and cholesterol binding sites in voltage-dependent anion channel-1 determined by photo-affinity labeling. Biochim. Biophys. Acta BBA—Mol. Cell Biol. Lipids 1864, 1269-1279 (2019).
54. Rovini, A. et al. Molecular mechanism of olesoxime-mediated neuroprotection through targeting α-synuclein interaction with mitochondrial VDAC. Cell. Mol. Life Sci. (2019) doi:10.1007/s00018-019-03386-w.
55. Ben-Hail, D. & Shoshan-Barmatz, V. VDAC1-interacting anion transport inhibitors inhibit VDAC1 oligomerization and apoptosis. Biochim. Biophys. ActaBBA—Mol. Cell Res. 1863, 1612-1623 (2016).
56. Ben-Hail, D. et al. Novel Compounds Targeting the Mitochondrial Protein VDAC1 Inhibit Apoptosis and Protect against Mitochondrial Dysfunction. J. Biol. Chem. 291, 24986-25003 (2016).
57. Haloi, N. et al. Structural Basis of Complex Formation Between Mitochondrial Anion Channel VDAC1 and Hexokinase-II. bioRxiv 2020.11.18.365965 (2020) doi:10.1101/2020.11.18.365965.
58. Horton K. L., Stewart K. M., Fonseca S. B., Guo Q., Kelley S. O. Mitochondria-penetrating peptides. Chem Biol. 2008 April; 15(4):375-82.

1. An HK-derived peptide comprising the amino acid sequence: alanine (A)-glutamine (Q)-X₁-X₂-X₃-tyrosine (Y)-tyrosine (Y)-X₄ (SEQ ID NO:1), wherein
X₁ is leucine (L) or tryptophan (W)
X₂ is leucine (L) or tryptophan (W)
X₃ is alanine (A), D-isomer alanine (A_{D}) or α-aminoisobutyric acid (U) and
X₄ is phenylalanine (F), leucine (L) or tyrosine (Y),
wherein the HK-derived peptide does not consist of the amino sequence set forth as SEQ ID NO:95 and the HK-derived peptide does not comprises the amino sequence set forth as SEQ ID NO:96.

2. The HK-derived peptide according to claim 1, comprising the amino acid sequence: alanine (A)-glutamine (Q)-X₁-X₂-X₃-tyrosine (Y)-tyrosine (Y)-X₄-threonine (T)-glutamic acid (E)-X₅-lysine (K) (SEQ ID NO:2), wherein
X₁ is leucine (L) or tryptophan (W)
X₂ is leucine (L) or tryptophan (W)
X₃ is alanine (A), D-isomer alanine (A_{D}) or α-aminoisobutyric acid (U)
X₄ is phenylalanine (F), leucine (L) or tyrosine (Y) and
X₅ is leucine (L) or tryptophan (W).

3. The HK-derived peptide according to claim 1, wherein X₃ is α-aminoisobutyric acid (U).

4. The HK-derived peptide according to claim 1, wherein the HK derived peptide of the invention comprises 8, 9, 10, 11, 12, 13, 14, 15 or 16 amino acids.

5. The HK-derived peptide according to claim 1, wherein the HK-derived peptide comprises or consists of the amino sequence selected in the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52 and SEQ ID NO:53.

6. The HK-derived peptide according to claim 5, wherein the HK-derived peptide comprises or consists of the amino sequence SEQ ID NO: 18, SEQ ID NO:20, SEQ ID NO:25, SEQ ID NO:26, SEQ ID NO:28 or SEQ DI NO:29.

7. The HK-derived peptide according to claim 1, wherein a sequence AUAU (SEQ ID NO:54) or a sequence AU (SEQ ID NO:55) is coupled to the HK-derived peptide.

8. The HK-derived peptide according to claim 1, wherein a dipeptide 3-CF₃Ph[Tz]U is coupled to N-terminal of the HK-derived peptide, wherein the dipeptide 3-CF₃Ph[Tz]U has the following formula:

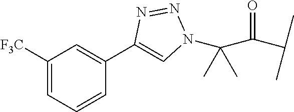 [SMILES: CC(C)C(=O)C(C)(C)n1cc(-c2cccc(C(F)(F)F)c2)nn1]

9. The HK-derived peptide according to, wherein a cell penetrating sequence is coupled to the HK-derived peptide.

10. The HK-derived peptide according to claim 9, wherein the cell penetrating sequence is tat (SEQ ID NO:58).

11. A vector encoding the HK-derived peptide according to claim 1.

12. (canceled)

13. A method for treating a peripheral demyelinating disease, a myocardium diseases, cancer, diabetes, a lupus-like disease, non-alcoholic fatty liver disease, or a neurogenerative disease in a subject in need thereof, comprising,
administering to the subject a therapeutically effective amount of the peptide of claim 1 or a vector encoding the peptide.

14. The method according to claim 13, wherein the peripheral demyelinating disease is selected from the group consisting of Refsum's disease, Abetalipoproteinemia, Tangier disease, Krabbe's disease, Metachromatic leukodystrophy, Fabry's disease, Dejerine-Sottas syndrome, Charcot-Marie-Tooth Disease, Hereditary Neuropathy with liability to pressure palsies (HNPP), Familial Amyloidotic Neuropathy, Hereditary sensory neuropathy Type II (HSN II), hereditary porphyria, muscular dystrophy, Dejerine-Sottas syndrome, diabetic neuropathy, an immune-mediated neuropathy, Acute Motor Neuropathy, Acute Sensory Neuropathy, Acute Autonomic Neuropathy, miller-fisher syndrome, Chronic Polyneuropathies, a peripheral demyelinating disease associated with vasculitis or inflammation of the blood vessels in peripheral nerves, a peripheral demyelinating diseases associated with monoclonal gammopathies, a peripheral demyelinating diseases associated with tumors or neoplasms, a peripheral demyelinating diseases caused by drugs, a peripheral demyelinating disease caused by infections, a peripheral demyelinating disease caused by nutritional imbalance, a peripheral demyelinating disease arising in kidney diseases, hypothyroid neuropathy, a peripheral demyelinating disease caused by alcohol and toxins, a peripheral demyelinating disease caused by trauma or compression, and an idiopathic peripheral demyelinating disease.

15. A pharmaceutical composition comprising the peptide according to claim 1 or a vector encoding the peptide.

16. (canceled)


SEQUENCE LISTING (96 sequences)

SEQ ID NO: 1 - protein, 8 aa, Artificial Sequence
  Synthetic sequence
  MISC_FEATURE (3)..(3): X= Leu or Trp
  MISC_FEATURE (4)..(4): X= Leu or Trp
  MISC_FEATURE (5)..(5): X= Ala, isomer-D Ala or Aib
  MISC_FEATURE (8)..(8): X= Phe, Leu or Tyr
AQXXXYYX

SEQ ID NO: 2 - protein, 12 aa, Artificial Sequence
  Synthetic SEQ ID NO2
  MISC_FEATURE (3)..(4): X= Leu or Trp
  MISC_FEATURE (4)..(4): X= Leu or Trp
  MISC_FEATURE (5)..(5): X= Ala, isomer-D Ala or Aib
  MISC_FEATURE (8)..(8): X= Phe, Leu or Tyr
  MISC_FEATURE (11)..(11): X= Leu or Trp
AQXXXYYXTEXK

SEQ ID NO: 3 - protein, 12 aa, Artificial Sequence
  Synthetic 5i
AQLLAYYLTEWK

SEQ ID NO: 4 - protein, 12 aa, Artificial Sequence
  Synthetic 5j
AQLLAYYYTEWK

SEQ ID NO: 5 - protein, 12 aa, Artificial Sequence
  Synthetic 5k
AQWLAYYFTEWK

SEQ ID NO: 6 - protein, 12 aa, Artificial Sequence
  Synthetic 5l
AQWLAYYLTELK

SEQ ID NO: 7 - protein, 12 aa, Artificial Sequence
  Synthetic 5m
AQWLAYYYTELK

SEQ ID NO: 8 - protein, 12 aa, Artificial Sequence
  Synthetic 5n
AQWLAYYLTEWK

SEQ ID NO: 9 - protein, 12 aa, Artificial Sequence
  Synthetic 5o
AQWLAYYYTEWK

SEQ ID NO: 10 - protein, 12 aa, Artificial Sequence
  Synthetic 5p
AQLWAYYFTEWK

SEQ ID NO: 11 - protein, 12 aa, Artificial Sequence
  Synthetic 5q
AQLWAYYLTELK

SEQ ID NO: 12 - protein, 12 aa, Artificial Sequence
  Synthetic 5r
AQLWAYYYTELK

SEQ ID NO: 13 - protein, 12 aa, Artificial Sequence
  Synthetic 5s
AQLWAYYLTEWK

SEQ ID NO: 14 - protein, 12 aa, Artificial Sequence
  Synthetic 5t
AQLWAYYYTEWK

SEQ ID NO: 15 - protein, 12 aa, Artificial Sequence
  Synthetic 5u
AQWWAYYFTELK

SEQ ID NO: 16 - protein, 12 aa, Artificial Sequence
  Synthetic 5v
AQWWAYYLTELK

SEQ ID NO: 17 - protein, 12 aa, Artificial Sequence
  Synthetic 5w
AQWWAYYYTELK

SEQ ID NO: 18 - protein, 12 aa, Artificial Sequence
  Synthetic 5x
AQWWAYYFTEWK

SEQ ID NO: 19 - protein, 12 aa, Artificial Sequence
  Synthetic 5y
AQWWAYYLTEWK

SEQ ID NO: 20 - protein, 12 aa, Artificial Sequence
  Synthetic 5z
AQWWAYYYTEWK

SEQ ID NO: 21 - protein, 11 aa, Artificial Sequence
  Synthetic 6l
  MOD_RES (1)..(1): Nle
XIAAQWLAYYL

SEQ ID NO: 22 - protein, 11 aa, Artificial Sequence
  Synthetic 6m
  MOD_RES (1)..(1): Nle
XIAAQWLAYYY

SEQ ID NO: 23 - protein, 11 aa, Artificial Sequence
  Synthetic 6n
  MOD_RES (1)..(1): Nle
XIAAQLWAYYL

SEQ ID NO: 24 - protein, 11 aa, Artificial Sequence
  Synthetic 6o
  MOD_RES (1)..(1): Nle
XIAAQLWAYYY

SEQ ID NO: 25 - protein, 11 aa, Artificial Sequence
  Synthetic 6p
  MOD_RES (1)..(1): Nle
XIAAQWWAYYF

SEQ ID NO: 26 - protein, 11 aa, Artificial Sequence
  Synthetic 6q
  MOD_RES (1)..(1): Nle
XIAAQWWAYYL

SEQ ID NO: 27 - protein, 11 aa, Artificial Sequence
  Synthetic 6r
  MOD_RES (1)..(1): Nle
XIAAQWWAYYY

SEQ ID NO: 28 - protein, 12 aa, Artificial Sequence
  Synthetic 7a′
  MOD_RES (5)..(5): Aib
AQLLXYYFTELK

SEQ ID NO: 29 - protein, 12 aa, Artificial Sequence
  Synthetic 7g′
  MOD_RES (5)..(5): Aib
AQWWXYYFTEWK

SEQ ID NO: 30 - protein, 12 aa, Artificial Sequence
  Synthetic 5i-U
  MOD_RES (5)..(5): Aib
AQLLXYYLTEWK

SEQ ID NO: 31 - protein, 12 aa, Artificial Sequence
  Synthetic 5j-U
  MOD_RES (5)..(5): Aib
AQLLXYYYTEWK

SEQ ID NO: 32 - protein, 12 aa, Artificial Sequence
  Synthetic 5k-U
  MOD_RES (5)..(5): Aib
AQWLXYYFTEWK

SEQ ID NO: 33 - protein, 12 aa, Artificial Sequence
  Synthetic 5l-U
  MOD_RES (5)..(5): Aib
AQWLXYYLTELK

SEQ ID NO: 34 - protein, 12 aa, Artificial Sequence
  Synthetic 5m-U
  MOD_RES (5)..(5): Aib
AQWLXYYYTELK

SEQ ID NO: 35 - protein, 12 aa, Artificial Sequence
  Synthetic 5n-U
  MOD_RES (5)..(5): Aib
AQWLXYYLTEWK

SEQ ID NO: 36 - protein, 12 aa, Artificial Sequence
  Synthetic 5o-U
  MOD_RES (5)..(5): Aib
AQWLXYYYTEWK

SEQ ID NO: 37 - protein, 12 aa, Artificial Sequence
  Synthetic 5p-U
  MOD_RES (5)..(5): Aib
AQLWXYYFTEWK

SEQ ID NO: 38 - protein, 12 aa, Artificial Sequence
  Synthetic 5q-U
  MISC_FEATURE (5)..(5): X=U
AQLWXYYLTELK

SEQ ID NO: 39 - protein, 12 aa, Artificial Sequence
  Synthetic 5r-U
  MOD_RES (5)..(5): Aib
AQLWXYYYTELK

SEQ ID NO: 40 - protein, 12 aa, Artificial Sequence
  Synthetic 5s-U
  MOD_RES (5)..(5): Aib
AQLWXYYLTEWK

SEQ ID NO: 41 - protein, 12 aa, Artificial Sequence
  Synthetic 5t-U
  MOD_RES (5)..(5): Aib
AQLWXYYYTEWK

SEQ ID NO: 42 - protein, 12 aa, Artificial Sequence
  Synthetic 5u-U
  MOD_RES (5)..(5): Aib
AQWWXYYFTELK

SEQ ID NO: 43 - protein, 12 aa, Artificial Sequence
  Synthetic 5v-U
  MOD_RES (5)..(5): Aib
AQWWXYYLTELK

SEQ ID NO: 44 - protein, 12 aa, Artificial Sequence
  Synthetic 5w-U
  MOD_RES (5)..(5): Aib
AQWWXYYYTELK

SEQ ID NO: 45 - protein, 12 aa, Artificial Sequence
  Synthetic 5y-U
  MOD_RES (5)..(5): Aib
AQWWXYYLTEWK

SEQ ID NO: 46 - protein, 12 aa, Artificial Sequence
  Synthetic 5z-U
  MOD_RES (5)..(5): Aib
AQWWXYYYTEWK

SEQ ID NO: 47 - protein, 11 aa, Artificial Sequence
  Synthetic 6l-U
  MOD_RES (1)..(1): Nle
  MOD_RES (8)..(8): Aib
XIAAQWLXYYL

SEQ ID NO: 48 - protein, 11 aa, Artificial Sequence
  Synthetic 6m-U
  MOD_RES (1)..(1): Nle
  MOD_RES (8)..(8): Aib
XIAAQWLXYYY

SEQ ID NO: 49 - protein, 11 aa, Artificial Sequence
  6n-U
  MOD_RES (1)..(1): Synthetic Nle
  MOD_RES (8)..(8): Aib
XIAAQLWXYYL

SEQ ID NO: 50 - protein, 11 aa, Artificial Sequence
  Synthetic 6o-U
  MOD_RES (1)..(1): Nle
  MOD_RES (8)..(8): Aib
XIAAQLWXYYY

SEQ ID NO: 51 - protein, 11 aa, Artificial Sequence
  Synthetic 6p-U
  MOD_RES (1)..(1): Nle
  MOD_RES (8)..(8): Aib
XIAAQWWXYYF

SEQ ID NO: 52 - protein, 11 aa, Artificial Sequence
  Synthetic 6q-U
  MOD_RES (1)..(1): Nle
  MOD_RES (8)..(8): Aib
XIAAQWWXYYL

SEQ ID NO: 53 - protein, 11 aa, Artificial Sequence
  Synthetic 6r-U
  MOD_RES (1)..(1): Nle
  MOD_RES (8)..(8): Aib
XIAAQWWXYYY

SEQ ID NO: 54 - protein, 4 aa, Artificial Sequence
  Synthetic SEQ ID NO54
  MOD_RES (2)..(2): Aib
  MOD_RES (4)..(4): Aib
AXAX

SEQ ID NO: 55 - protein, 2 aa, Artificial Sequence
  Synthetic SEQ ID NO55
  MOD_RES (2)..(2): Aib
AX

SEQ ID NO: 56 - protein, 16 aa, Artificial Sequence
  Synthetic Penetratin
RQIKIWFQNRRMKWKK

SEQ ID NO: 57 - protein, 8 aa, Artificial Sequence
  Synthetic R8
RRRRRRRR

SEQ ID NO: 58 - protein, 12 aa, Artificial Sequence
  Synthetic tat
GRKKRRQRRRPQ

SEQ ID NO: 59 - protein, 27 aa, Artificial Sequence
  Synthetic Transportan
GWTLNSAGYLLGKINLKALAALAKKIL

SEQ ID NO: 60 - protein, 7 aa, Artificial Sequence
  Synthetic Xentry
LCLRPVG

SEQ ID NO: 61 - protein, 8 aa, Artificial Sequence
  Synthetic Horton Peptide
  MOD_RES (1)..(1): Cha
  MOD_RES (3)..(3): Cha
  MOD_RES (5)..(5): Cha
  MOD_RES (7)..(7): Cha
XRXRXRXR

SEQ ID NO: 62 - protein, 15 aa, Artificial Sequence
  Synthetic 1a
LIAAQLLAYYFTELK

SEQ ID NO: 63 - protein, 15 aa, Artificial Sequence
  Synthetic 1b
AIAAQLLAYYFTELK

SEQ ID NO: 64 - protein, 15 aa, Artificial Sequence
  Synthetic 1c
LAAAQLLAYYFTELK

SEQ ID NO: 65 - protein, 15 aa, Artificial Sequence
  Synthetic 1d
LIAAALLAYYFTELK

SEQ ID NO: 66 - protein, 15 aa, Artificial Sequence
  Synthetic 1e
LIAAQLAAYYFTELK

SEQ ID NO: 67 - protein, 15 aa, Artificial Sequence
  Synthetic 1f
LIAAQLAAYYFTELK

SEQ ID NO: 68 - protein, 15 aa, Artificial Sequence
  Synthetic 1g
LIAAQLLAAYFTELK

SEQ ID NO: 69 - protein, 15 aa, Artificial Sequence
  Synthetic 1h
LIAAQLLAYAFTELK

SEQ ID NO: 70 - protein, 15 aa, Artificial Sequence
  Synthetic 1i
LIAAQLLAYYATELK

SEQ ID NO: 71 - protein, 15 aa, Artificial Sequence
  Synthetic 1j
LIAAQLLAYYFAELK

SEQ ID NO: 72 - protein, 15 aa, Artificial Sequence
  Synthetic 1k
LIAAQLLAYYFTALK

SEQ ID NO: 73 - protein, 15 aa, Artificial Sequence
  Synthetic 1l
LIAAQLLAYYFTEAK

SEQ ID NO: 74 - protein, 15 aa, Artificial Sequence
  Synthetic 1m
LIAAQLLAYYFTELA

SEQ ID NO: 75 - protein, 14 aa, Artificial Sequence
  Synthetic 3a
IAAQLLAYYFTELK

SEQ ID NO: 76 - protein, 13 aa, Artificial Sequence
  Synthetic 3b
AAQLLAYYFTELK

SEQ ID NO: 77 - protein, 12 aa, Artificial Sequence
  Synthetic 3c
AQLLAYYFTELK

SEQ ID NO: 78 - protein, 10 aa, Artificial Sequence
  Synthetic 3d
QLAYYFTELK

SEQ ID NO: 79 - protein, 10 aa, Artificial Sequence
  Synthetic 3e
LLAYYFTELK

SEQ ID NO: 80 - protein, 9 aa, Artificial Sequence
  Synthetic 3f
LAYYFTELK

SEQ ID NO: 81 - protein, 8 aa, Artificial Sequence
  Synthetic 3g
AYYFTELK

SEQ ID NO: 82 - protein, 14 aa, Artificial Sequence
  Synthetic 4a
LIAAQLLAYYFTEL

SEQ ID NO: 83 - protein, 13 aa, Artificial Sequence
  Synthetic 4b
LIAAQLLAYYFTE

SEQ ID NO: 84 - protein, 12 aa, Artificial Sequence
  Synthetic 4c
LIAAQLLAYYFT

SEQ ID NO: 85 - protein, 11 aa, Artificial Sequence
  Synthetic 4d
LIAAQLLAYYF

SEQ ID NO: 86 - protein, 10 aa, Artificial Sequence
  Synthetic 4e
LIAAQLLAYY

SEQ ID NO: 87 - protein, 9 aa, Artificial Sequence
  Synthetic 4f
LIAAQLLAY

SEQ ID NO: 88 - protein, 8 aa, Artificial Sequence
  Synthetic 4g
LIAAQLLA

SEQ ID NO: 89 - protein, 8 aa, Artificial Sequence
  Synthetic SEQ ID NO89
AQLLAYYF

SEQ ID NO: 90 - protein, 28 aa, Artificial Sequence
  Synthetic Wt HK1 peptide
MIAAQLLAYYFTELKGRKKRRQRRRPPQ

SEQ ID NO: 91 - protein, 28 aa, Artificial Sequence
  Synthetic CMT4G-mutated HK1 peptide
MGQICQRESATAAEKGRKKRRQRRRPPQ

SEQ ID NO: 92 - protein, 13 aa, Artificial Sequence
  Synthetic Control peptide
GRKKRRQRRRPPQ

SEQ ID NO: 93 - protein, 917 aa, Homo sapiens
MIAAQLLAYYFTELKDDQVKKIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVNEYSLNAGKQRYEKMISGMYLGEIVRNILIDFTKKGFLFRGQISETMKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 94 - protein, 923 aa, Homo sapiens
MGQICQRESATAAEKPKLHLLAESEHCIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVNEYSLNAGKQRYEKMISGMYLGEIVRNILIDFTKKGFLFRGQISETMKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 95 - protein, 8 aa, Homo sapiens
AQLLAYYF

SEQ ID NO: 96 - protein, 13 aa, Homo sapiens
AAQLLAYYFTELK